SEQUENCE LISTING (19 sequences)

SEQ ID NO: 1 - protein, 27 aa, Acyrthosiphon pisum
MIFFKQYSMMITFIVIAVWVMPAITSE

SEQ ID NO: 2 - protein, 25 aa, Acyrthosiphon pisum
MVFYKQYLLTITCIVITAWVIPTSA

SEQ ID NO: 3 - protein, 25 aa, Acyrthosiphon pisum
MVFFKQYLITLTCIVISVWITPVNT

SEQ ID NO: 4 - protein, 25 aa, Acyrthosiphon pisum
MIFFKQYLIILTFIVIAVLVMPVTP

SEQ ID NO: 5 - protein, 25 aa, Acyrthosiphon pisum
MVFFKQYLLTLTCIVIVVQVMPASA

SEQ ID NO: 6 - protein, 25 aa, Acyrthosiphon pisum
MIFFKQYLIMLTFIIIAVWVMPANT

SEQ ID NO: 7 - protein, 23 aa, Acyrthosiphon pisum
MSFFKQYLTLTFIVISVWNMSEA

SEQ ID NO: 8 - protein, 19 aa, Acyrthosiphon pisum
MVFFKQFLITLTVIIITEA

SEQ ID NO: 9 - protein, 25 aa, Acyrthosiphon pisum
MVFFKLYLLTLTCIVIAVWVMPVSA

SEQ ID NO: 10 - protein, 53 aa, Acyrthosiphon pisum
MFNVLIILSLISYTFEPSYTLYKFKMVFFKQDLLMLTCITIAVWIMPPSASTN

SEQ ID NO: 11 - protein, 19 aa, Acyrthosiphon pisum
MVFFRQFLITLSVILITEA

SEQ ID NO: 12 - protein, 26 aa, Acyrthosiphon pisum
MHFFKHYLIVLTYIVISFWFMPSASL

SEQ ID NO: 13 - protein, 22 aa, Acyrthosiphon pisum
MFKHIIVLVLCFMAYFVGNLDA

SEQ ID NO: 14 - protein, 22 aa, Acyrthosiphon pisum
MDKHIIMLALCLMVYIIGNIDA

SEQ ID NO: 15 - protein, 22 aa, Acyrthosiphon pisum
MLKHIIVLALYLMAYIIGNIDA

SEQ ID NO: 16 - protein, 20 aa, Acyrthosiphon pisum
MLKHILLALCFMAYIIENIG

SEQ ID NO: 17 - protein, 21 aa, Acyrthosiphon pisum
MLKHILLALCFMAYIIENIGA

SEQ ID NO: 18 - protein, 18 aa, Acyrthosiphon pisum
MLKHIIVLVLCFMPYIIG

SEQ ID NO: 19 - protein, 23 aa, Acyrthosiphon pisum
MGSYKLYVAVMAIAIAVVQEVRC

(19) United States
(12) Patent Application Publication
(10) Pub. No.: US 2025/0034654 A1
(43) Pub. Date: Jan. 30, 2025

(54) IDENTIFICATION OF BICYCLE GENES

(71) Applicant: HOWARD HUGHES MEDICAL INSTITUTE, Chevy Chase, MD (US)

(72) Inventors: David L. Stern, Leesburg, VA (US); Clair Han, Toronto (CA)

(21) Appl. No.: 18/681,891

(22) PCT Filed: Sep. 14, 2022
(86) PCT No.: PCT/US2022/043503

§ 371 Date: Feb. 7, 2024

Related U.S. Application Data
(60) Provisional application No. 63/243,904, filed on Sep. 14, 2021.

(51) Int. Cl. C12Q 1/6888; C12Q 1/6874; G16B 20/20

(52) U.S. Cl. CPC C12Q 1/6888; C12Q 1/6874; G16B 20/20; C12Q 2600/124; C12Q 2600/158

(57) ABSTRACT

A method of identifying a bicycle gene involves determining for a candidate gene a series of gene structure-based predictor variables, and applying a bicycle gene classifier including the predictor variables to determine whether the candidate gene is identified as a bicycle gene. The gene structure-based predictor variables can be selected from the following: (i) total gene length (base pair, bp); (ii) total length (bp) of coding exons; (iii) first coding exon length (bp); (iv) last coding exon length (bp); (v) number of internal exons in phase 0); (vi) number of internal exons in phase 1; (vii) number of internal exons in phase 2; and (viii) mean internal exon length (bp).

### RELATED APPLICATIONS

This application is a 371 application of International Patent Application No. PCT/US2022/043503 filed Sep. 14, 2022, which claims priority from U.S. Provisional Application Ser. No. 63/243,904 filed Sep. 14, 2021, the entire disclosure of which is incorporated herein by this reference.

### REFERENCE TO AN ELECTRONIC SEQUENCE LISTING

The contents of the electronic sequence listing (Stern-2021-032-02 ST26.xml; Size: 17,825 bytes; and Date of Creation: Nov. 2, 2022) is herein incorporated by reference in its entirety.

### TECHNICAL FIELD

The presently-disclosed subject matter generally relates to methods for the identification of bicycle genes, and nucleic acid molecules, polypeptide molecules, and compositions that can be identified and/or generated using such identification methods. In particular, certain embodiments of the presently-disclosed subject matter relate to methods for the identification of bicycle genes that are based on gene structure, and are coding sequence-independent.

### INTRODUCTION

One challenge that is faced by evolutionary and functional genetic studies is that many genes have been identified as “orphan” or lineage-specific genes because homologous sequences cannot be identified outside of a limited taxonomic range. Many lineage-specific genes may not be truly novel, however, since sequence divergence can cause homologs to become undetectable by sequence-search methods (Vakirlis et al., 2020); Weisman et al., 2020). Identifying such extremely divergent homologs remains a significant bioinformatic challenge and limits functional inferences derived from homology (Loewenstein et al., 2009).

Genes involved in host-parasite systems often evolve extremely divergent sequences as a result of genetic conflict (Eizaguirre et al., 2012; Paterson et al., 2010). Aphids and their host plants represent one such antagonistic pair. Aphids are small insects that feed by inserting their thin mouthparts (stylets) into the phloem vessels of plants to extract nutrients (Dixon, 1978). Like many herbivorous insects, aphids introduce effector molecules into plant tissues to manipulate the physiology and development of plants to the insects' advantage (Mutti et al., 2008; Hogenhout and Bos, 2011; Elzinga and Jander, 2013; Rodriguez et al., 2017). For example, aphids introduce calcium binding proteins that prevent the plant's ability to block phloem cell transport in response to phloem vessel damage (Will et al., 2007). It is believed that aphids introduce a wide range of effector proteins and that these molecules contribute to the debilitating effects of multiple aphid species on plants, which imposes significant financial damage on most major agricultural crops worldwide (Schaible et al., 2008).

In an extreme form of aphid manipulation of plant physiology and development, some aphid species induce novel plant organs, called galls, which provide the aphids with a ready food source and with protection from the elements and from natural enemies (Giron et al., 2016; Shorthouse et al., 2005). Aphid Galls can be significant nutrient sinks (Burstein et al., 1994), demonstrating that galling aphids induce both local and long-range changes to plant physiology.

Many arthropods induce galls on a wide diversity of plant species (Mani, 1964), but the complex life cycle of galling aphids provides unique advantages for studying the mechanisms of gall induction that are not found in other galling arthropods. In most galling aphid species, only one generation each year, the gall foundress or fundatrix, appears to be capable of inducing galls. Individuals of subsequent generations in the life cycle do not induce galls, even though they may feed on the same host plant. Comparisons of genes transcribed specifically in salivary glands of individuals across generations provided some of the first clues to the putative effector proteins contributing to gall induction (Korgaonkar et al., 2021).

The genome of one species of galling aphid encodes 476 related genes encoding diverse presumptive effector proteins with no sequence homology to previously described proteins and that many of these proteins contain two cysteine-tyrosine-cysteine (CYC) motifs (Korgaonkar et al., 2021). These genes were therefore named bicycle (bi-CYC-like) genes. A genome-wide association study revealed that variation in one bicycle gene, called determinant of gall color (dgc), was strongly associated with a red versus green gall color polymorphism and this genetic polymorphism was associated with strong downregulation of dgc in aphids and strong upregulation of a small number of genes involved in anthocyanin production in plant galls. In addition, the majority of bicycle genes are strongly upregulated in the salivary glands specifically of the aphid generation that induces galls (the fundatrix). Together with genetic evidence that dgc regulates the gall phenotype, the enrichment of bicycle genes expressed in fundatrix salivary glands suggested that many bicycle genes may contribute to gall development or physiology.

The primary amino-acid sequences of Bicycle proteins have evolved rapidly, apparently in response to extremely strong positive selection (Korgaonkar et al., 2021). In preliminary studies, there were attempts to identify bicycle homologs in other insect species using sequence similarity algorithms such as Basic Local Alignment Search Tool (BLAST) (Altschul et al., 1990) and hmmer (Eddy, 2011; Johnson et al., 2010), but identified few putative homologs. It was therefore unclear if bicycle genes are a recently evolved family of genes or an ancient family that has evolved highly divergent coding sequences.

Accordingly, there is a need in the arty for a system for a sequence-independent method for identifying bicycle genes, for purposes of exploring the evolutionary history of bicycle genes, and for use in various applications, such as directed modification of cells.

### SUMMARY

The presently-disclosed subject matter meets some or all of the above-identified needs, as will become evident to those of ordinary skill in the art after a study of information provided in this document.

Extremely divergent homologs have been identified previously using features of gene structures that evolve more slowly than the coding sequence, such as exon sizes and intron positions (Bazan, 1991; Brown et al., 1995; Betts, 2001), and these characteristics of genes have also proven to be valuable for phylogeny reconstruction (Rokas et al., 1999; Venkatesh et al., 1999; Rokas and Holland, 2000; Telford and Copley, 2011). Additionally, it was noted that bicycle genes have unusual gene structures containing many micro-exons (Korgaonkar et al., 2021) and, in developing the subject matter disclosed herein, the possibility that bicycle gene structure may be more conserved than bicycle gene sequences was considered.

The presently-disclosed subject matter includes a method that allows for identification of highly divergent bicycle gene homologs that does not rely on sequence similarity. A logistic regression classifier is used, based only on structural features of bicycle genes. This method was tested and found to be very accurate, despite the fact that it does not include any sequence information.

A method of the presently-disclosed subject matter for identifying a bicycle gene involves determining for a candidate gene a series of gene structure-based predictor variables, and applying a bicycle gene classifier including the predictor variables to determine whether the candidate gene is identified as a bicycle gene. The gene structure-based predictor variables can be selected from the following: (i) total gene length (base pair, bp); (ii) total length (bp) of coding exons; (iii) first coding exon length (bp); (iv) last coding exon length (bp); (v) number of internal exons in phase 0; (vi) number of internal exons in phase 1; (vii) number of internal exons in phase 2; and (viii) mean internal exon length (bp).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1. The gene structure-based classifier detects bicycle genes in genomes of aphids, phylloxerids, and coccids. A whole-proteome phylogeny of the species studied is shown on the left. Family membership is shown in color beside the species names. The number of candidate bicycle homologs detected by BLAST, hmmer, and the gene structure-based classifier is shown to the right of the phylogeny. A second hmmer search was performed starting with the candidate bicycle homologs identified by the gene structure-based classifier within each species, and the results of this search are shown in the far right column. Scale bar represents 0.1 substitutions per residue.

FIG. 2A-2H. Salient features of gene structure of H. cornu bicycle genes. FIG. 2A. Sequence similarity between H. cornu bicycle genes versus the distance between genes. Most of the most similar bicycle genes are located within 100 kb of each other, supporting a model of frequent tandem or local duplication. However, many similar genes are located on different chromosomes, suggesting that bicycle genes often transpose to different chromosomes. FIG. 2B. The proportion of the forty flanking (20 upstream and 20 downstream) orthologous genes surrounding bicycle genes or randomly selected genes between H. cornu and T. nigriabdominalis. Far fewer homologous genes are found flanking bicycle orthologs than between randomly selected orthologs, which suggests that conserved synteny is not a highly informative measure of bicycle gene homology. FIG. 2C-2D. Almost all internal exons of bicycle genes are of phase 2 (FIG. 2C), whereas the majority of internal exons of other genes in the H. cornu genome are of phase 0 (FIG. 2D). FIG. 2E-2F. Bicycle genes have a wide range of exon numbers, with a distribution (FIG. 2E) different from non-bicycle genes (FIG. 2F). FIG. 2G-2H. (G-H) The total length of Bicycle proteins is better predicted by the number of exons in the gene (FIG. 2G) than by the length of the first and last exon (FIG. 2H).

FIG. 3A-3J. Performance of a gene-structure based classifier of bicycle genes. FIG. 3A. Gene structure features used in classifier. The intron-exon structure of a generic gene is shown at the top. The six features tested in the classifier are illustrated below the gene. FIG. 3B. Precision versus recall curves for replicate training runs of the classifier using 100 different subsets of the training data. The curves across all subsets are very similar, indicating that the classifier was not unduly influenced by a specific subset of the data. FIG. 3C. Histogram of the number of transcripts with a particular classifier response shows a bimodal distribution. The vast majority of genes (>10,000) had a response close to 0. A threshold of 0.72 was chosen at a precision to recall ratio of 1. FIG. 3D. Precision (the proportion of true positives among both true and false positives) versus recall (the proportion of true positives among true positives and false negatives) for the model trained on 476 previously identified H. cornu bicycle genes. Differently shaded dots represent the precision to recall ratio for different probability cutoffs of the model, shown in FIG. 3D. FIG. 3E. The ratio of precision to recall for different cutoffs. Precision approximately equals recall (P/R=1) at a cutoff of 0.72. FIG. 3F. Precision and recall values for models where one variable at a time was removed from the model. Removal of the number of exons with mode2 had the largest decrement on performance, but all models performed well. FIG. 3G. Precision and recall values for models where only one predictor variable was included in the model. Exon mean length had the best predictive value on its own, but no single variable performed as well as inclusion of all variables. FIG. 3H. Correlation amongst predictor variables for all of the original 476 bicycle genes. Most variables are not strongly correlated. FIG. 3I. First two principal components of principal component analysis of values for the predictor variables for all of the original 476 bicycle genes provides further support that variables are not strongly correlated. FIG. 3J. Percent variance explained for the eight principal components of a principal component analysis for the predictor variables for all of the original 476 bicycle genes.

FIG. 4. Plots of exon number per gene vs median exon size for all species studied. Phylogeny for all species is shown on left and is identical to phylogeny in FIG. 1. Bicycle genes tend to have many small exons. Red=bicycle classifier, blue=other genes.

FIG. 5A-5H. The gene-structure based classifier identifies additional CYC and non-CYC motif containing genes in the H. cornu genome that may contribute to the effector protein repertoire of multiple stages of the H. cornu life cycle. FIG. 5A. Hierarchical clustering of candidate H. cornu bicycle homologs detected by the gene-structure based on amino-acid sequence similarity measure reveals two clusters of genes with similar sequences. Some of the genes in Cluster 1 appear to have some sequence similarity to Cluster 2 genes. FIG. 5B. Genes in Cluster 1 encode proteins with N-terminal signal sequences and weak similarity to bicycle genes. FIG. 2C. Genes in Cluster 2 encode proteins with sequence similarity to the previously described bicycle genes. FIG. 5D. Diagram of life cycle of H. cornu. Individuals from different generations exhibit phenotypes that are specialized for each stage of the complex life cycle. The fundatrix (G1) is the only generation that induces a gall. This panel was published previously as FIG. 1J in Korgaonkar et al. (2001) and is available under Creative Commons Attribution License (CC BY 4.0). FIG. 5E. A volcano plot of the strength of evidence for differential expression (−log 10(P value)) versus the fold-change differential expression of salivary glands isolated from fundatrices versus sexuals reveals that some of the Cluster1 (black) and 2 (orange) genes identified by the gene structure based classifier are as strongly enriched in fundatrix salivary glands as the originally described bicycle genes (blue). In addition, several Cluster 1 and 2 genes are more strongly enriched in salivary glands of sexuals. FIG. 5F-5G. Plots of gene expression levels for Cluster 1 (FIG. 5F) and 2 (FIG. 5G) genes across four generations of the life cycle reveals that while most bicycle homologs are most strongly expressed in fundatrix salivary glands (e.g. purple lines), some are weakly expressed in fundatrix salivary glands and then strongly expressed in salivary glands of individuals from other stages of the life cycle (e.g. red lines).

FIG. 6A-6D. Newly identified candidate bicycle genes in H. cornu exhibit signatures of strong and recent positive selection. FIG. 6A-6C. d_{N}/dₛ ratio for the 476 original bicycle genes (FIG. 6A), the newly identified putative bicycle genes (FIG. 6B) and the non-bicycle genomic background (FIG. 6C) between H. cornu and H. hamamelidis. Both the original bicycle genes and the new putative bicycle genes show an excess of genes with a ratio of _{dN/ds} above 1 compared to the background. Dashed vertical line indicates d_{N}/dₛ=1. FIG. 6D. Median distance to the closest sweep signal for original bicycle genes (red dashed line) and classifier newly identified putative bicycle genes (blue dashed line). Histogram shows median distance to closest sweep signal for 1000 permutations of gene labels. The original bicycle genes and the new putative bicycle genes are both closer to sweep signals than expected by chance.

FIG. 7A-7E. H. cornu bicycle genes belong to four major categories, unicycle, bicycle, tricycle, and tetracycle genes. FIG. 7A. The originally identified bicycle genes encode proteins that exhibit a wide size range, with representatives of approximately four size classes, indicated by the vertical dotted blue lines. FIG. 7B-E. Logo plots of the proteins from each of the four size classes of Bicycle proteins identifies unicycle (FIG. 7B), bicycle (FIG. 7C), tricycle (FIG. 7D), and tetracycle (FIG. 7E) proteins. The CYC motifs in each protein are identified by thick black lines below each Logo plot.

FIG. 8A-8D. Gene-structure aware alignments reveal extensive conservation of intron locations between the originally-defined bicycle genes and putative bicycle homologs identified by the gene-structure based classifier. Predicted proteins encoded by the original or newly identified bicycle homologs were divided by sequence length into unicycle (FIG. 8A), bicycle (FIG. 8B), tricycle (FIG. 8C), or tetracycle (FIG. 8D) proteins and aligned using gene-structure aware alignment. The frequency of introns at each location in the alignment is plotted for the original (orange) and newly defined (cyan) bicycle homologs and positions without introns were excluded from the plot. Intron positions are therefore relative to each other and do not indicate absolute position in the proteins or the alignments. Coincidence of intron positions is shown by purple bars. Original bicycle homologs were randomly down-sampled to 20 genes to match the number of newly identified bicycle homologs to improve overall alignment accuracy (FIG. 8B).

FIG. 9A-9E. Testing intron concordance. To determine whether concordant introns were found more often than expected by chance alignment of unrelated genes, gene-structure aware alignment was performed between a subset of the original bicycle genes and genes from three gene families containing at least 10 genes with at least 10 exons each: SLC33A1, abcG23, and nrf-6. Intron concordance was measured as the correlation coefficient between the number of genes exhibiting an intron in the test gene family versus the original bicycle gene family. FIG. 9A. Example of gene-structure aware alignment of 23 genes from SLC33A1 and 23 original bicycle gene families. One intron was found frequently in both gene families, a pattern observed in most alignments and likely reflects an artifact of the gene-structure aware alignment algorithm. FIG. 9B. Example of gene-structure aware alignment of 20 candidate H. cornu bicycle genes identified by the classifier and 20 original bicycle genes. Many introns are found in both groups of genes. FIG. 9C-9D. Examples of quantification of intron concordance, measured as the correlation between the number of genes with an intron at each location in the alignment in the test gene family versus the original bicycle genes. For a comparison of the unrelated gene families SLC33AJ and original bicycle genes (FIG. 9C), the correlation was close to 0. For a comparison of candidate bicycle genes versus the original bicycle genes, the correlation was high and positive (FIG. 9D). FIG. 9E. Intron concordance—measured as the correlation coefficient (R) of the number of genes sharing intron locations in gene-structure aware alignments—of repeated re-samplings of unrelated genes families generated a null distribution of expected R values for unrelated genes (SLC33A1, abcG23, and nrf-6 versus original bicycle genes in red, green and blue histograms, respectively). R values for all comparisons of candidate bicycle genes found by the classifier and the original bicycle genes were all positive and much higher than any of the R values resulting from alignment of unrelated gene families, indicating that more introns are shared between genes in these gene-structure aware alignments than expected between unrelated gene families by chance (P<0.01).

FIG. 10A-10F. The T. nigriabdominalis bicycle homologs detected by the gene structure-based classifier display CYC motifs and highly divergent protein sequences. FIG. 10A. Hierarchical clustering of candidate T. nigriabdominalis bicycle homologs based on amino-acid sequence similarity measure reveals four clusters of genes. FIG. 10B-10E. The four clusters of T. nigriabdominalis bicycle homologs display two (FIG. 10B, 10C) or one (FIG. 10D) CYC motif, or no clear CYC motifs (FIG. 10E). The cluster identities are indicated in each subplot. (FIG. 10F) Volcano plot of genes differentially expressed in fundatrix salivary glands versus carcass with bicycle genes labelled. Most of the T. nigriabdominalis candidate bicycle homologs are strongly over-expressed in the fundatrix salivary glands.

FIG. 11A-11C. A. pisum bicycle homologs contain a single CYC motif and are strongly enriched in salivary glands. FIG. 11A. Logo plot of A. pisum bicycle homologs detected by gene structure-based classifier. Most A. pisum homologs contain a single CYC motif. FIG. 11B. Volcano plot of differential expression between salivary glands and carcass reveals that A. pisum bicycle homologs are strongly over-expressed in salivary glands. FIG. 11C. The A. pisum putative bicycle homologs share multiple intron positions with H. cornu bicycle genes. Proteins were aligned using gene-structure aware alignment and the frequency of introns at each location in the alignment was plotted in different colors for the two classes of genes and positions without introns were excluded from the plot. Intron positions are therefore relative to each other and do not indicate absolute position in the proteins or the alignments. Sites of coincident introns are indicated in purple.

FIG. 12A-12C. Tetraneura nigriabdominalis life cycle and additional features of T. nigriabdominalis bicycle genes. FIG. 12A. Photo of mature galls of T. nigriabdominalis on leaf of Ulmus americana collected at Janelia Research Campus, Ashbum, VA, USA. FIG. 12B. Diagram of life cycle of T. nigriabdominalis. Samples for differential expression analysis were collected from generations G1 and G2. FIG. 12C. The T. nigriabdominalis putative bicycle homologs from Clusters 1 and 4, with and without CYC motifs, respectively, share multiple intron positions. Proteins from the two classes of genes were aligned using gene-structure aware alignment and the frequency of introns at each location in the alignment was plotted in different colors for the two classes of genes and positions without introns were excluded from the plot. Intron positions are therefore relative to each other and do not indicate absolute position in the proteins or the alignments.

FIG. 13A-13F. Features of bicycle homologs detected in aphid species. FIG. 13A-13B. The number of bicycle homologs detected by the gene-structure based classifier versus are not correlated with genome N50 (A; R2=0.09, F=1.4, P=0.26) nor with the number of genes annotated for each genome (B; R2=0.002, F=0.4, P=0.69). FIG. 13C-13F. Logo plots of putative bicycle homologs detected in four aphid species, M persicae (FIG. 13C), P. nigronervosa (FIG. 13D), R. maidis (FIG. 13E), and C. cedri (FIG. 13F) reveals that they all contain CYC motifs. The M persicae and P. nigronervosa genes are primarily unicycles, the R. maidis genes are primarily bicycles, and the C. cedri genes are primarily tetracycles.

FIG. 14A-14F. Features of bicycle homologs detected in outgroups to aphids. FIG. 14A-14C. Logo plots of proteins encoded by putative bicycle homologs detected in D. vitilfoliae (FIG. 14A), M. hirsutus (FIG. 14B), and P. solenopsis (FIG. 14C). The logo plots from these putative homologs show no obvious CYC motifs. FIG. 14D-14F. Gene-structure aware alignments reveal that multiple introns are shared between these highly divergent putative bicycle homologs and H. cornu bicycle genes. Positions in alignments without introns were excluded from the plot. Intron positions are therefore relative to each other and do not indicate absolute position in the proteins or the alignments. Sites of coincident introns are indicated in purple.

FIG. 15A-15E. Megacycle genes have a repeating protein structure and are overexpressed in salivary glands of multiple aphid species. FIG. 15A. Gene structure of the M. sacchari megacycle gene (g20694). The gene includes 118 exons encoded in approximately 100 kbp of DNA. The protein is 2092 AA long. FIG. 15B. Rapid automatic detection and alignment of repeats (RADAR) analysis of the M. sacchari megacycle gene provides evidence for 15 repeating units within the protein encoded by g20694. Multiple sequence alignment of these repeat units (not using information on gene structure) reveals that many introns, marked in green, are located in identical or similar positions in the aligned sub-sequences. A logo plot of the aligned repeated sequences is shown above the alignment. There is no obvious evidence for CYC motifs, although the motif is approximately the same length as the proteins encoded by many bicycle genes. FIG. 15C-15E. Volcano plots of differential expression between salivary glands and carcasses for three species, H. cornu (FIG. 15C), T. nigriabdominalis (FIG. 15D), and A. pisum (FIG. 15E), reveals that megacycle genes (black dots) are strongly over-expressed in salivary glands of all three species. Bicycle genes are marked as light grey dots.

FIG. 16A-16B. Megacycle genes share intron locations with bicycle genes and are found in many aphid species. FIG. 16A. Gene-structure aware alignments reveal that multiple introns are shared between megacycle genes and H. cornu bicycle genes. Positions in alignments without introns were excluded from the plot. Intron positions are therefore relative to each other and do not indicate absolute position in the proteins or the alignments. FIG. 16B. Maximum likelihood phylogeny of the protein sequences encoded by megacycle genes found in aphid species. Megacycle genes were not detected outside of aphids. The phylogeny has a topology similar to the aphid portion of the whole-proteome phylogeny shown in FIG. 1. Only one megacycle gene was detected in most aphid species, but three paralogs were detected in C. cedri and T. nigriabdominalis. Values at nodes are bootstrap support values. Scale bar is 1 substitution per residue.

FIG. 17A-17D. The classifier predicted 770 bicycle genes when applied to the published genome and transcriptome data for S. chinensis. Logo plots were provided (FIG. 17A-17C) for each of three size classes groups of predicted bicycle genes, which were divided into these three group based on their length (FIG. 17D).

FIG. 18A-18B. Predicted bicycle genes from S. chinensis share more intron locations with bicycle genes of H. cornu than expected by chance. Sequences were aligned with an intron-aware alignment method and shared intron positions were identified (FIG. 18A). The correlation coefficient (R) between the number of introns from each species at each position is shown as the dashed line in FIG. 18B. The distribution of correlation coefficients for control comparisons of unrelated genes are provided (FIG. 18B).

FIG. 19. Plot of gene expression levels of predicted bicycle genes across the following generations: AM—Autumn Migrant, FM—Sexual Female, FU—Generation 1—Fundatrix, GL—Generation 2—Fundatriginae, L1—Nymph, L2—Nymph, MA—Sexual Male, MF—Pregnant Female, and SE—Spring Migrant.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes methods for the identification of bicycle genes. The presently-disclosed subject matter further includes methods for confirmation or validation of identified bicycle genes.

The presently-disclosed subject matter further includes nucleic acid molecules, polypeptide molecules, and compositions that can be identified and/or generated using such identification methods. Embodiments of the presently-disclosed subject matter relate to methods for the identification of bicycle genes that are based on gene structure, and are not dependent on coding sequence.

In some embodiments, the presently disclosed subject matter includes a method of identifying a bicycle gene, which involves determining for a candidate gene a series of gene structure-based predictor variables, and applying a bicycle gene classifier including the predictor variables to determine whether the candidate gene is identified as a bicycle gene.

The gene structure-based predictor variables can be selected from the following: (i) total gene length (base pair, bp); (ii) total length (bp) of coding exons; (iii) first coding exon length (bp); (iv) last coding exon length (bp); (v) number of internal exons in phase 0; (vi) number of internal exons in phase 1; (vii) number of internal exons in phase 2; and (viii) mean internal exon length (bp).

Multiple predictor variables are employed to identify a bicycle gene according to the method disclosed herein. In this regard, in some embodiments, two, three, four, five, six, or more predictor variable are employed. In certain preferred embodiments, seven or eight predictor variables are employed. In some embodiments, multiple predictor variables are employed, wherein one of the predictor variables is the number of internal exons in phase 2. In some embodiments, the following seven predictor variables are employed: (i) total gene length (base pair, bp); (ii) total length (bp) of coding exons; (iii) first coding exon length (bp); (iv) last coding exon length (bp); (v) number of internal exons in phase 0; (vi) number of internal exons in phase 1; and (vii) number of internal exons in phase 2. In some embodiments, all eight of the predictor variables as identified herein are employed.

In some embodiments of the method of identifying a bicycle gene as disclosed herein, the bicycle gene classifier is a generalized linear model (GLM). In some embodiments, a series of coefficients can be used in connection with the GLM, including an intercept coefficient, as well as coefficients to the predictor variables. Depending on the application, useful coefficients can include those obtained within a desired confidence interval, e.g., 99.9% (Z=3.291), 99.5% (Z=2.807), 99% (Z=2.576), 95% (Z=1.960), 90% (Z=1.645), 85% (Z=1.440), or 80% (Z=1.282) confidence interval. In some embodiments, the coefficients can be selected from within the following ranges: intercept=1.63 to 10.9, total exon length=−0.0333 to −0.00795, total gene length=−0.000126 to −0.00000235, first exon length=−0.0232 to 0.0105, last exon length=−0.00654 to 0.0272, mean exon length=−0.143 to 0.0119, number of exons in phase 0=−4.87 to −1.77, phase 1=−2.74 to 0.5, and phase 2=0.622 to 1.79.

The presently-disclosed subject matter also includes a method of identifying a polypeptide encoded by a bicycle gene, which involves identifying a bicycle gene according to the method described herein, and determining the amino acid sequence encoded by the nucleotide sequence of the bicycle gene. In this manner, the amino acid sequence of the polypeptide is identified and can be prepared and/or modified as desired using methods known to those of ordinary skill in the art. Accordingly, the presently disclosed subject matter further includes a polypeptide, comprising the amino acid sequence of the polypeptide identified according to the disclosed method. The presently disclosed subject matter further includes a composition comprising such polypeptide.

The presently-disclosed subject matter also includes a polynucleotide, comprising the sequence of a bicycle gene identified according to the method described herein. Once a candidate gene sequence is identified as a bicycle gene, its nucleotide sequence can be prepared and/or modified as desired using methods known to those of ordinary skill in the art. Accordingly, the presently disclosed subject matter further includes such a polynucleotide. The presently disclosed subject matter further includes a vector comprising such polynucleotide. The presently disclosed subject matter further includes a composition comprising such vector.

The presently-disclosed subject matter also includes a polypeptide that includes one, two, three, or four cysteine-tyrosine-cysteine (CYC) motifs, attached to one or more flexible or rigid linkers that separate and connect CYC motifs when two or more CYC motifs are present.

The presently-disclosed subject matter also includes a method of modifying a cell, which involves administering to the cell a polynucleotide, polypeptide, composition, or vector as disclosed herein. In embodiments of the method, the cell can be, for example, a plant cell, an animal cell, a fungal cell, or a bacterial cell.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

### Example 1. Gene Structure Based Bicycle Gene Classifier

The bicycle gene classifier was built as a generalized linear model (GLM) with the following predictor variables: total gene length, total length of coding exons, first coding exon length, last coding exon length, mean internal exon length, and number of internal exons in phase 0, 1, and 2. All length measurements are in base pairs. All genes with zero or one internal exon were removed from the training and test datasets because mean internal exon lengths and number of internal exons starting in each of the three phases cannot be accurately estimated; hence, the classifier was unable to detect any potential bicycle genes with only one to three exons. The dependent variable from the GLM is the predictor of bicycle gene classification. The GLM was implemented with the glm function as binomial with logit link, and response variables for the whole-genome gene set were predicted using predict, both in the R base package stats v.3.6.1.

In the training set, the dependent variable of the GLM is whether the transcript was previously classified as a bicycle gene (Korgaonkar et al., 2021), where bicycle genes were coded as 1 and non-bicycle genes were coded as 0. Only genes classified as either bicycle genes or previously annotated genes were used in the training set due to the possibility that unannotated genes include additional previously undetected bicycle genes. Previously annotated genes were identified as those having at least one significant match at E<0.01 after Bonferroni correction for multiple searches with blastp (blast v.2.9.0), blastx (blast v.2.9.0), or hmmscan (HMMER v3.2.1) against the Pfam database.

The performance of the classifier was evaluated using a precision-recall curve where

$$precision = \frac{T P}{T P + F P} and recall = \frac{T P}{T P + F N} ,$$

where TP, FP, and FN are the number of true positives, false positives, and false negatives, respectively. To determine a cutoff value on the dependent variable of the GLM for bicycle gene classification, all possible cutoffs from 0 to 0.98 were tested in increments of 0.02 to find the cutoff resulting in a precision to recall ratio closest to 1. The model was first validated by repeatedly sampling and training on 70% of the data and testing on the remaining 30% of the data for 100 replicates and found no substantial change to the trained classifier or the resulting cutoff value (mean 0.7338, s.d. 0.1578) amongst replicates.

The final classifier was trained on all H. cornu bicycle genes and annotated non-bicycle genes without subsampling and determined a cutoff value of 0.72 (precision=0.9925, recall=0.9925). The coefficients to the independent variables in the GLM are as follows: intercept=6.26, total exon length=−0.0206, total gene length=−0.0000642, first exon length=−0.00632, last exon length=0.0103, mean exon length=−0.0655, number of exons in phase 0=−3.32, phase 1=−1.12, and phase 2=1.21. The contribution of each independent variable was evaluated by training the H. cornu dataset using GLMs with each term on its own and each term removed one at a time from the full model. The GLM trained by the H. cornu dataset was applied to twenty-one other species belonging to the families of Aphidoidae, Phylloxeroidae, Coccoidea, Psylloidea, Aleyrodoidea, and Fulgoroidea.

Useful coefficients can also include those obtained within a 95% confidence interval (Z=1.96), which are as follows: intercept=1.63 to 10.9, total exon length=−0.0333 to −0.00795, total gene length=−0.000126 to −0.00000235, first exon length=−0.0232 to 0.0105, last exon length=−0.00654 to 0.0272, mean exon length=−0.143 to 0.0119, number of exons in phase 0=−4.87 to −1.77, phase 1=−2.74 to 0.5, and phase 2=0.622 to 1.79.

Depending on the application, useful coefficients can include those obtained within a desired confidence interval, e.g., 99.9% (Z=3.291), 99.5% (Z=2.807), 99% (Z=2.576), 95% (Z=1.960), 90% (Z=1.645), 85% (Z=1.440), or 80% (Z=1.282) confidence interval.

### Example 2: Aphid Collections and Dissections

Aphids of T. nigriabdominalis were collected from galls collected on Ulmus americana on the grounds of Janelia Research Campus (Table 1, Table 2). Species identification was confirmed using both morphological characteristics (Walczak et al., 2019) and by comparing the mitochondrial DNA of the genome, we assembled with the mitochondrial sequences at NCBI available for many species of Tetraneura (Lee and Akimoto, 2015; Hebert et al., 2016).

Aphids of A. pisum LSR1 were kindly provided by Greg Davis and grown on broad bean plants in the laboratory. Salivary glands were dissected out of multiple instars of fundatrices and their offspring for T. nigriabdominalis and of virginoparae for A. pisum and stored in 3 μL of Smart-seq2 lysis buffer (0.2% Triton X-100, 0.1 U/mL RNasin Ribonuclease Inhibitor) at −80° C. for later mRNA isolation. Carcasses of both species were ground in PicoPure extraction buffer and mRNA was prepared using the PicoPure mRNA extraction kit. RNAseq libraries were prepared as described previously (Korgaonkar et al., 2021).

TABLE 1
Details of Tetraneura nigriabdominalis sample, sequence
files, and SRA accession numbers for genome sequencing. JRC
refers to Janelia Research Campus, Ashburn VA, USA. These
sequences can be found under the SRA BioProject PRJNA759586

| | | Library | | | |
|---|---|---|---|---|---|
| Collection | | Prep | Sequencing | # Read | |
| Location | Species | Method | Method | pairs | Accession # |
| JRC | Tetraneura | 10X | Illumina | 441,975,717 | SRR15704183 |
| | nigriabdominalis | Chromium | PE151 | | |

TABLE 2
Details of biological samples, sequence files, and SRA accession numbers for
Tetraneua nigriabdominalis RNA-sequencing. JRC refers to Janelia Research Campus,
Ashburn VA. These sequences can be found under the SRA BioProject PRJNA762862.

| | | | | | Library | | | |
|---|---|---|---|---|---|---|---|---|
| | Collection | Collection | | | Prep | Sequencing | # | Accession |
| Sample ID | Date | Location | Generation | Organ | Method | Method | Reads | # |
| Tnig_Fund_{—} | Apr. 22, 2019 | JRC | 1 | SG | WGB | Illumina | 8475508, | SRR18396612 |
| SG_2 | | | | | | PE150 | 5308849 | |
| Tnig_Fund_{—} | Apr. 22, 2019 | JRC | 1 | SG | WGB | Illumina | 6087930, | SRR18396611 |
| SG_4 | | | | | | PE150 | 6250323 | |
| Tnig_Fund_{—} | Apr. 22, 2019 | JRC | 1 | SG | WGB | Illumina | 7474183, | SRR18396610 |
| SG_5 | | | | | | PE150 | 7147731 | |
| Tnig_Fund_{—} | Apr. 22, 2019 | JRC | 1 | Carcass | WGB | Illumina | 6208251, | SRR18396609 |
| Carc_2 | | | | | | PE150 | 6194859 | |
| Tnig_Fund_{—} | Apr. 22, 2019 | JRC | 1 | Carcass | WGB | Illumina | 7453030, | SRR18396608 |
| Carc_4 | | | | | | PE150 | 6095776 | |
| Tnig_Fund_{—} | Apr. 22, 2019 | JRC | 1 | Carcass | WGB | Illumina | 8399343, | SRR18396607 |
| Carc_5 | | | | | | PE150 | 7332616 | |
| Tnig_2ndGen | Apr. 22, 2019 | JRC | 2 | SG | WGB | Illumina | 6902051, | SRR18396606 |
| SG 2 1 | | | | | | PE150 | 7037142 | |
| Tnig_2ndGen_{—} | Apr. 22, 2019 | JRC | 2 | SG | WGB | Illumina | 6888742, | SRR18396605 |
| SG_2_2 | | | | | | PE150 | 6997123 | |
| Tnig_2ndGen_{—} | Apr. 22, 2019 | JRC | 2 | SG | WGB | Illumina | 6049169, | SRR18396604 |
| SG_2_3 | | | | | | PE150 | 6696157 | |

### Example 3: Genome Sequencing, Assembly, and Annotation of Tetraneura Nigriabdominalis

HMW gDNA was prepared from T. nigriabdominalis aphids isolated from a single gall following the Salting Out Method provided by 10× Genomics (support.10×genomics.com/genome-exome/sample-prep/doc/demonstrated-protocol-salting-out-method-for-dna-extraction-from-cells). 10× linked-read libraries were prepared and Chromium sequencing was performed by HudsonAlpha Institute for Biotechnology. Genomes were assembled with Supernova v.2.1.0 commands run and mkoutput (Weisenfeld et al., 2017).

133,280,000 reads were used for an estimated raw genome coverage of 56.18×. The genome size of the assembled scaffolds was 344.312 Mb and the scaffold N50 was 10 Mb. Using BUSCO completeness analysis with gVolante version 1.2.0 and BUSCO_v2/v3 (Nishimura et al., 2017), the genome was found to contain 1035 of 1066 (97.1%) completely detected core arthropod genes and 1043 of 1066 (97.8%) partially detected core genes. The genome was annotated for protein-coding genes using BRAKER (Hoff et al., 2019) with multiple RNA-seq libraries (Table 2).

This genome has been deposited at GenBank under the accession JAIUCT000000000.

### Example 4: Differential Expression Analysis

Candidate bicycle genes from H. cornu, T. nigriabdominalis, and A. pisum identified by the classifier were manually re-annotated in Apollo (Dunn et al., 2019) guided by RNAseq data viewed in Integrative Genomics Viewer (IGV) (Thorvaldsdóttir et al., 2013). Samples used for analysis are described in Tables 2-4. Differential expression analyses were performed as described previously (Korgaonkar et al., 2021) using edgeR v3.28.1, and full analysis scripts are provided at doi.org/10.25378/janelia.17868413, doi.org/10.25378/janelia.17892089, and doi.org/10.25378/janelia.17912180, respectively.

TABLE 3
Details of biological samples, sequence files, and SRA accession numbers for Acyrthosiphon
pisum RNA-sequencing. The aphid strain used was LSR1 (Caillaud et al., 2002), originally
collected in the vicinity of Ithaca, NY, USA in summer 1998. Aphids were grown on broad
bean plants in the laboratory at Janelia Reseach Campus and dissected for these samples
on 21 Feb. 2021. Sequences can be found under SRA BioProject PRJNA762703.

| | | | | | Library | | | |
|---|---|---|---|---|---|---|---|---|
| | Collection | Collection | | | Prep | Sequencing | # | Accession |
| Sample ID | Date | Location | Generation | Organ | Method | Method | Reads | # |
| SG1 | 1998 | Ithaca, NY, | 1 | SG | WGB | Illumina | 1273082, | SRR15862168 |
| | | USA | | | | PE150 | 5724222 | |
| SG2 | 1998 | Ithaca, NY, | 1 | SG | WGB | Illumina | 1557673, | SRR15862167 |
| | | USA | | | | PE150 | 6606339 | |
| SG3 | 1998 | Ithaca, NY, | 1 | SG | WGB | Illumina | 1199959, | SRR15862166 |
| | | USA | | | | PE150 | 5221105 | |
| SG4 | 1998 | Ithaca, NY, | 1 | SG | WGB | Illumina | 1159521, | SRR15862165 |
| | | USA | | | | PE150 | 5169430 | |
| carc1 | 1998 | Ithaca, NY, | 1 | Carcass | WGB | Illumina | 998879, | SRR15862164 |
| | | USA | | | | PE150 | 4363153 | |
| carc2 | 1998 | Ithaca, NY, | 1 | Carcass | WGB | Illumina | 1185062, | SRR15862163 |
| | | USA | | | | PE150 | 5433749 | |
| carc3 | 1998 | Ithaca, NY, | 1 | Carcass | WGB | Illumina | 1045749, | SRR15862162 |
| | | USA | | | | PE150 | 4901408 | |

TABLE 4
SRA accession numbers for Genomes downloaded from NCBI. Manually corrected
BRAKER annotations are available on Figshare at 10.25378/janelia. 17777888.

| | Original genomes | | | |
|---|---|---|---|---|
| | Genbank assembly | Publication | | |
| | accession or | source of each | Manually corrected | TRINITY |
| Species | alternative location | genome | BRAKER Annotation | assembly |
| Acyrthosiphon | bipaa.genouest.org/sp/ | (Mathers et al., | Apis.Augustus.updated_{—} | apis.Trinity.fasta.gz |
| pisum | acyrthosiphon_pisum/ | 2021) | w_annots.22xii21.gff3 | |
| | download/genome/JIC1_1.0/ | | | |
| Aphis gossypi | GCF_004010815.1 | (Quan et al., | Agos.Augustus.updated_{—} | agos.Trinity.fasta.gz |
| | | 2019) | w_annots.6ix21.gff3 | |
| Bemisia tabaci | GCA_001854935.1 | (Chen et al., | Btab.Augustus.updated_{—} | |
| | | 2016) | w_annots.7vii21.gff3 | |
| Cinara cedri | GCA_902439185.1 | (Julca et al., | Cced.Augustus.updated_{—} | cced.Trinity.fasta.gz |
| | | 2020) | w_annots.7vii21.gff3 | |
| Daktulosphaira | bipaa.genouest.org/sp/ | (Rispe et al., | Dvit.Augustus.updated_{—} | |
| vitifoliae | daktulosphaira_vitifoliae/ | 2020) | w_annots.6ix21.gff3 | |
| | download/genome/v3.1/ | | | |
| | Dv_mitochondrial_genome.fa | | | |
| Diaphorina citri | GCA_000475195.1 | (Saha et al., | Dcit.Augustus.updated_{—} | |
| | | 2017) | w_annots.7vii21.gff3 | |
| Diuraphis noxia | GCA_001186385.1 | (Nicholson et | Dnox.Augustus.updated_{—} | dnox.Trinity.fasta.gz |
| | | al., 2015) | w_annots.7vii21.gff3 | |
| | | (Zhang et al., | | |
| | | 2014) | | |
| Ericerus pela | GCA_011428145.1 | (Yang et al., | Epel.Augustus.updated_{—} | |
| | | 2019) | w_annots.7vii21.gff3 | |
| Eriosoma | GCA_013282895.1 | (Biello et al., | Elan.Augustus.updated_{—} | elan.Trinity.fasta.gz |
| lanigerum | | 2021) | w_annots.7vii21.gff3 | |
| Hormaphis | GCA_017140985.1 | (Korgaonkar | Augustus.updated_w_{—} | hcor.Trinity.fasta.gz |
| cornu | | et al., 2021) | annots.19viii21_final.gff | |
| Laodelphax | GCA_003335185.2 | (Zhu et al., | Lstr.Augustus.updated_{—} | |
| striatella | | 2017) | w_annots.7vii21.gff3 | |
| Maconellicoccus | GCA_003261595.1 | (Kohli et al., | Mhir.Augustus.updated_{—} | |
| hirsutus | | 2021) | w_annots.6ix21.gff3 | |
| Melanaphis | GCA_002803265.2 | data.nal.usda.gov/ | Msac.Augustus.updated_{—} | msac.Trinity.fasta.gz |
| sacchari | | dataset/ | w_annots.7vii21.gff3 | |
| | | melanaphis- | | |
| | | sacchari- | | |
| | | strainlsu-and- | | |
| | | endosymbiont- | | |
| | | genome- | | |
| | | sequencing- | | |
| | | and- assembly | | |
| Myzus persicae | bipaa.genouest.org/sp/ | (Mathers et al., | Mper.Augustus.updated_{—} | mper.Trinity.fasta.gz |
| | myzus_persicae/ | 2021) | w_annots.13xii21.gff3 | |
| | download/genome/v2.0/ | | | |
| Nilaparvata | GCA_000757685.1 | (Xue et al., | Nlug.Augustus.updated_{—} | |
| | | 2014) | w_annots.6ix21.gff3 | |
| Pachypsylla | GCA_012654025.1 | (Y. Li et al., | Pven.Augustus.updated_{—} | |
| venusta | | 2020) | w_annots.7vii21.gff3 | |
| Pentalonia | GCA_014851325.1 | (Mathers et al., | Pnig.Augustus.updated_{—} | pnig.Trinity.fasta.gz |
| nigronervosa | | 2020) | w_annots.6ix21.gff3 | |
| Phenacoccus | GCA_009761765.1 | (M. Li et al., | Psol.Augustus.updated_{—} | |
| solenopsis | | 2020) | w_annots.6ix21.gff3 | |
| Rhopalosiphum | GCF_003676215.2 | (Chen et al., | Rmai.Augustus.updated_{—} | rmai.Trinity.fasta.gz |
| maidis | | 2019) | w_annots.6ix21.gff3 | |
| Sipha flava | GCA_003268045.1 | data.nal.usda.gov/dataset/ | Sfla.Augustus.updated_{—} | sfla.Trinity.fasta.gz |
| | | crop- | w_annots.6ix21.gff3 | |
| | | rotational- | | |
| | | diversity- | | |
| Tetraneura | JAIUCT000000000 | NA | Tnig.Augustus.updated_{—} | Tnig.Trinity.fasta.g |
| nigriabdominali | | | w_annots.7vii21.gff3 | |
| Trialeurodes | GCA_011764245.1 | (Xie et al., | Tvap.Augustus.updated_{—} | |
| vaporariorum | | 2020) | w_annots.7vii21.gff3 | |

### Example 5: Hmmer and BLAST Searches

The phylogeny of all species studied here was estimated using Orthofinder v 2.3.1 (Emmis and Kelly, 2018) on the complete gene sets predicted by BRAKER for all genomes using the following settings: -M msa -S diamond -A mafft -T fasttree. The phylogeny was plotted using ggtree v. 2.0.4 in R.

Bicycle homologs were searched for in all aphid species and outgroups using Position-Specific Interactive Basic Local Alignment Search Tool (PSI-BLAST) (Altschul, 1997) and hmmer (Eddy, 2011). PSI-BLAST v. 2.9.0+ was run using -max_target_seqs 5 and all other parameters as default. A cutoff of E<0.01 was used, Bonferroni corrected for 476 genes used as query searches. Profile-based hmmsearch v.3.2.1 was run using all default parameters and cutoff of E<0.01 without multiple testing correction.

### Example 6: Selection Tests

DnDs between H. cornu and H. hamamelidis for all transcripts in the transcriptome was calculated using the codeml function from the PAML package v4.9j (Yang, 2007). The genome-wide selection scan was performed using SweeD-P v.3.1 (Pavlidis et al., 2013). Details of both analyses can be found in the Methods section of Korgaonkar et al., 2021.

### Example 7: Gene-Structure Aware Sequence Alignment

Gene structure aware alignment was performed using prrn5 v 5.2.0 (Gotoh, 2021) with default weighting parameters. Gene-structure-informed extended fasta files were prepared that were suitable as input to prrn5 using the anno2gsiseq.pl utility provided at github.com/ogotoh/prm_aln/blob/master/perl/anno2gsiseq.pl. The results of these multiple sequence alignments were summarized by generating histograms with a bin size of 1 to illustrate the fraction of sequences in the alignment that had an intron at each position in the alignment.

To determine whether intron positions were shared between genes more often than expected by chance, we computed the concordance in intron positions resulting from prrn5 alignment of groups of unrelated genes. Paralog groups were identified for the whole H. cornu transcriptome using Orthofinder v 2.3.3 (Emms and Kelly, 2018) and identified three paralog groups containing at least 10 genes and having at least 10 exons for comparison with bicycle genes: SLC33A1, abcG23, and nrf-6. Intron concordance was measured as the correlation coefficient between the number of genes from the test gene family and the bicycle gene family containing an intron at each location in the alignment.

### Example 8: Re-Annotation of Previously Sequenced Genomes

The P. venusta genome is an approximately full-chromosome genome assembly that was kindly provided to the present inventors by Yiyuan Li and Nancy Moran (Li et al., 2020, 2019) prior to publication. All other genomes, except for H. cornu and T. nigriabdominalis, were downloaded from NCBI (Table 4). RNAseq data was downloaded from NCBI (Table 3) and predicted coding genes using BRAKER (Hoff et al., 2019) with the same workflow previously used in the discovery of bicycle genes (Korgaonkar et al., 2021).

### Example 9: Sequence-Based Homology Searches Find Few Bicycle Genes Outside of H. cornu

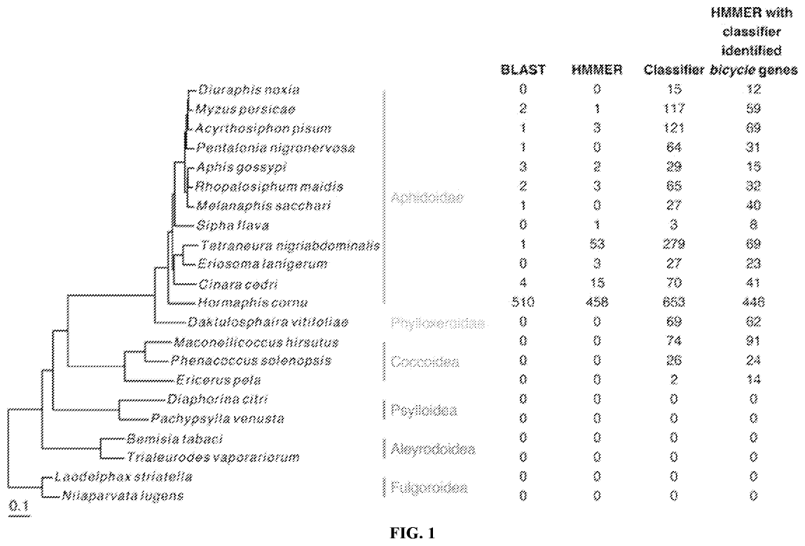

Bicycle genes in H. cornu were identified originally as a subset of previously unannotated genes that were enriched in the salivary gland mRNA of the gall-inducing foundress (Korgaonkar et al., 2021). Bicycle genes were found to be the single largest group of such genes that shared sequence similarity. These 476 genes are extremely divergent from one another at the amino-acid sequence level and appear to have evolved rapidly due to positive natural selection. Using sequence-based homology search methods BLAST and hmmer, 34 additional candidate bicycle paralogs were identified in H. cornu (using BLAST) and some putative homologs in other aphid species (in total 15 with BLAST and 81 with hmmer). No candidate bicycle orthologs were detected in genomes from insects of the families Phylloxeridae, Coccoidea, Psylloidea, Aleyrodoidea, and Fulgoroidea (FIG. 1).

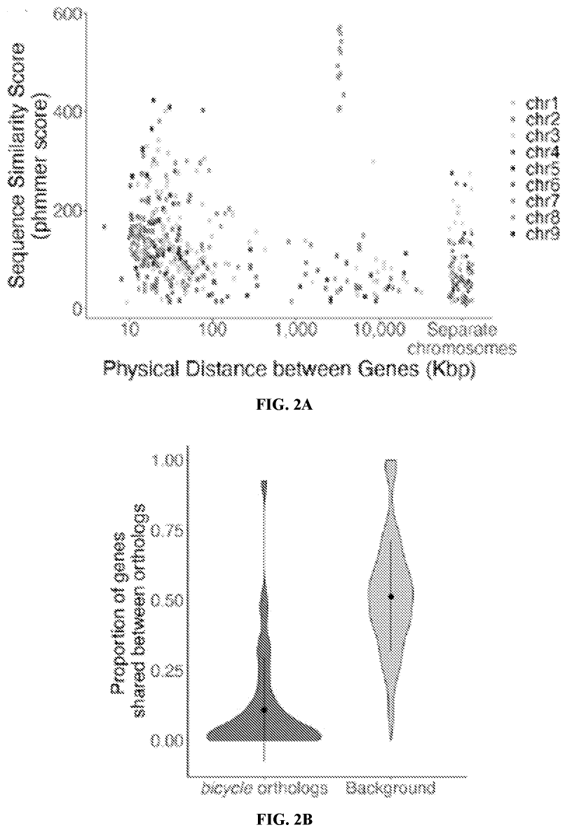

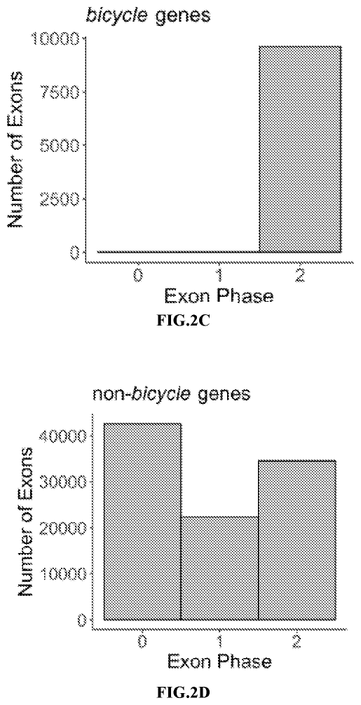

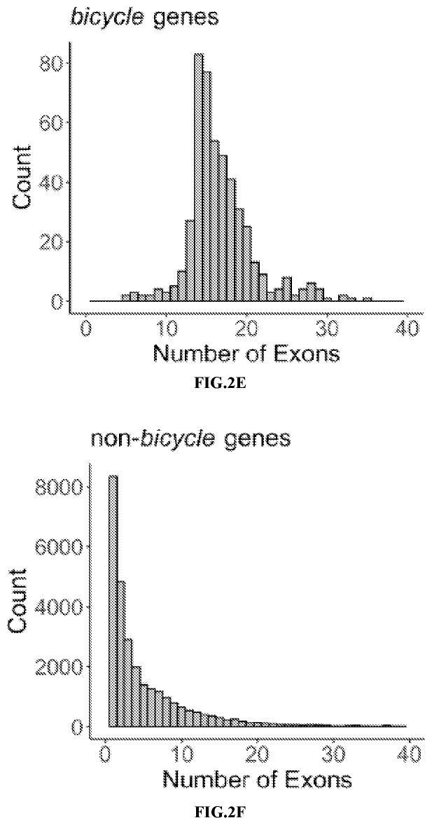

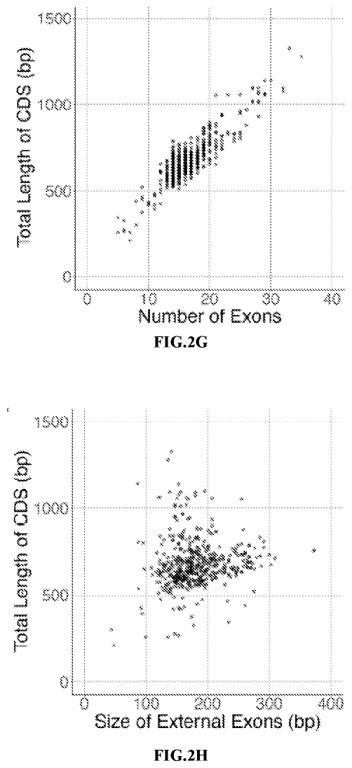

Gene synteny conservation can be used to identify highly divergent orthologs (Tekaia, 2016). Within the H. cornu genome, many closely related bicycle genes were located on separate chromosomes (FIG. 2A), suggesting that bicycle genes undergo transposition at a high rate and that bicycle genes are unlikely to display extensive conserved synteny. To more directly test whether conserved gene synteny may allow identification of bicycle homologs across the species studied here, we searched for conserved gene synteny near orthologous bicycle genes that were identified between H. cornu and Tetraneura nigriabdominalis (FIG. 1) as reciprocal best phmmer hits. Bicycle orthologs shared many fewer homologous genes in their flanking regions than randomly selected subsets of genes (FIG. 2B), further suggesting that conserved synteny is unlikely to be a useful measure to identify bicycle homologs across the species studied here. A different method is needed to identify highly divergent bicycle homologs.

### Example 10: A Classifier Based on Only Gene Structural Features can Identify Bicycle Genes with High Probability

Sequence-based search methods suggested that bicycle genes may be found in other species, but the low number of genes identified, and the incongruence between results from BLAST and hmmer suggested that sequence-based search may provide low sensitivity to detect bicycle homologs (Vakirlis et al., 2020; Weisman et al., 2020). Notably, while sequence divergence was high at the amino-acid level among H. cornu bicycle genes, non-sequence specific features of bicycle genes appeared to be relatively well conserved. For example, bicycle genes contain a large number of unusually small internal exons (Korgaonkar et al., 2021), making them clear outliers to most genes in the H. cornu genome. Also, almost all internal exons of bicycle genes were observed to start with codon position 2, that is they exhibit exon phase 2 (FIG. 2C), which is an extremely different distribution of exon phases compared with other genes in the genome (FIG. 2D). Genes containing many exons of similar size all with the same phase are rare in most genomes (Ruvinsky and Watson, 2007).

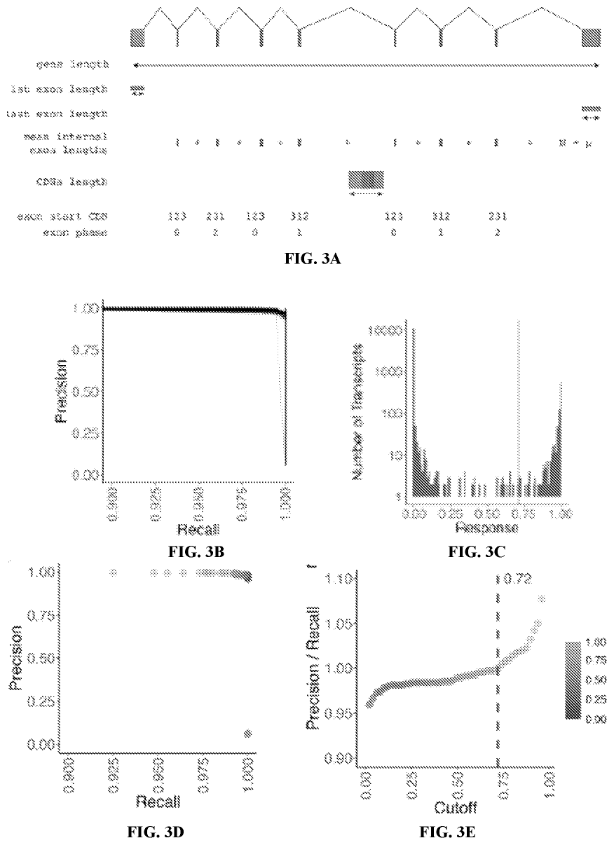

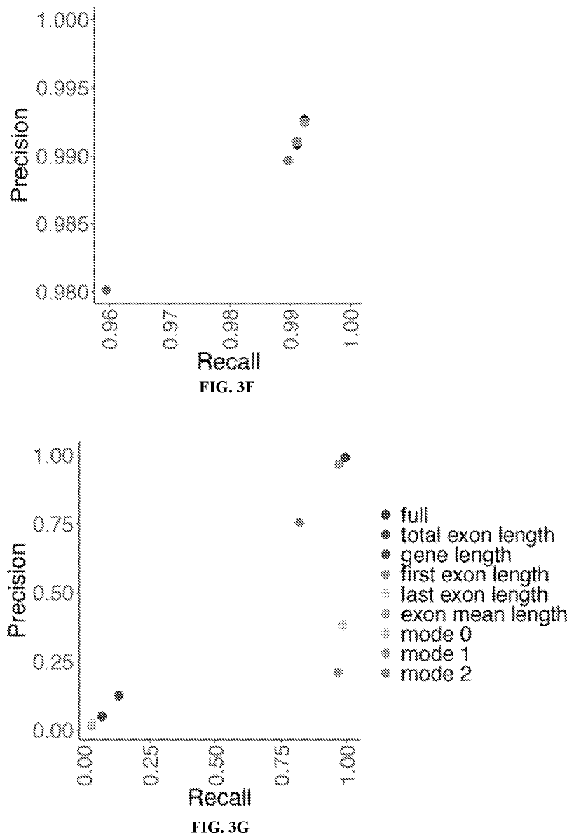

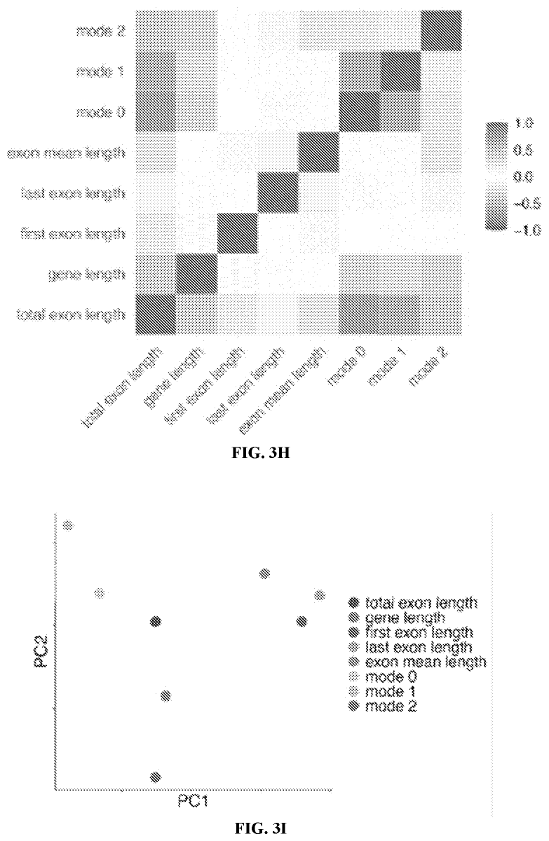

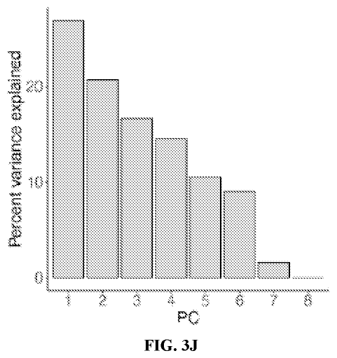

It was hypothesized that some aspects of bicycle gene structure may evolve more slowly than the primary protein sequences and thus provide a an evolutionary signal to detect distantly related bicycle homologs (Aguiar et al., 2015). For example, the pattern of exon phases can allow identification of highly-divergent homologous genes (Roy and Gilbert, 2005; Ruvinsky and Watson, 2007). Whether a logistic regression classifier could identify bicycle homologs based on the following structural features of each gene was explored: length of gene in genome, from start to stop codons; first and last exon length; internal exon mean length; length of the sum of codons; and number of internal exons that start in phase 0, 1, or 2 (FIG. 3A). Since almost all internal bicycle exons are of phase 2, there is no additional information from the order of exon phases in bicycle genes, and therefore the number of exons of each phase were simply counted.

Characterized bicycle genes have an average of approximately 17 and a minimum of 5 exons (FIG. 3E), which is strongly different from the genome-wide background (FIG. 3F, (two-sample Kolmogorov-Smimov test P-value<2.2×10-16)), and Bicycle protein lengths are well explained by total exon number (c.f. FIGS. 2G and 2H). Predicted genes containing at least four exons were searched to allow for the accurate summarization of internal exon mean lengths and number of internal exons that start in each of the three phases. Thus, if any bicycle genes have evolved to have three of fewer exons, then they could not be detected.

The classifier exhibited extremely good performance, with all data partitions (FIG. 3B). Therefore, a full model was constructed using all bicycle and annotated genes. (Non-annotated genes were excluded from model training and validation because it was hypothesized that this set may include additional bicycle homologs.)

The classifier categorized the vast majority of genes as either non-bicycle or bicycle genes with high probability (FIG. S3C). To decide on a probability cutoff to identify putative bicycle homologs, the trade-off between precision (the proportion of true positives among both true and false positives) and recall (the proportion of true positives among true positives and false negatives) was considered. A model probability cutoff that equalized precision and recall (P/R=1) was selected, which yielded precision and recall values of 0.99 (FIGS. 3D and 3E).

To determine whether all gene features were required for accurate classification, one factor at a time was removed, and the model was retrained. Precision and recall values were calculated using the same threshold as the full model. No single factor was solely necessary for model performance (FIG. 3F), although removal of the number of mode 2 exons resulted in the strongest decrement in model performance, resulting in precision and recall values of 0.98 and 0.96, respectively.

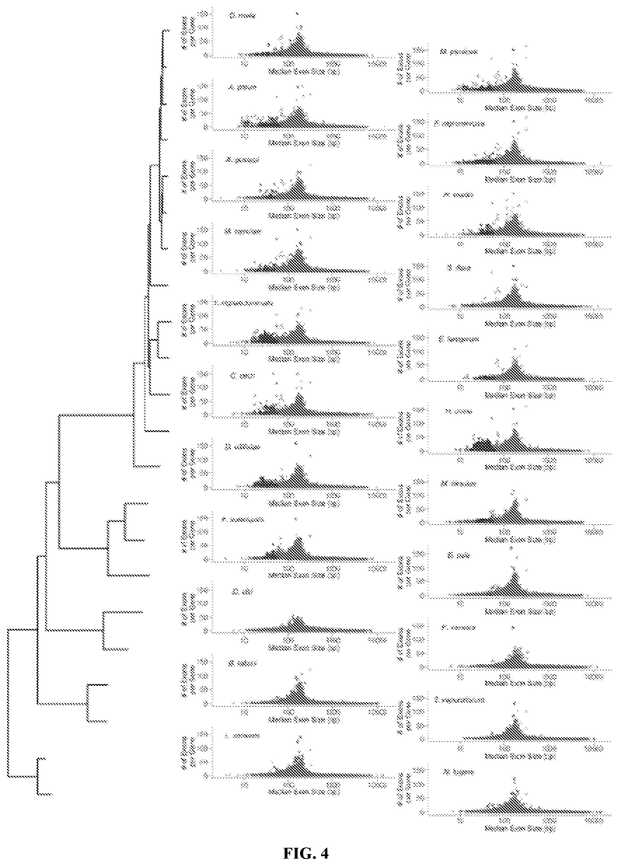

To determine whether any single predictor was sufficient to identify bicycle genes with high confidence, precision and recall values were calculated for linear models using one predictor at a time (FIG. 3G). Mean internal exon length alone performed best (FIG. 3G), consistent with the observation that bicycle genes contain an unusually large number of small exons (FIG. 4). The number of internal exons in mode 2 also displayed some predictive power on its own (FIG. 3G). No other predictors alone had substantial predictive power for discriminating bicycle genes from the background (FIG. 3G). The observation that no single predictor was solely necessary for performance but two predictors were individually sufficient to provide good predictive power indicates some correlation between the predictor variables (FIG. 3H-3J). Since no predictors were scalar multiples of each other, the full model using all predictors was used to search for new bicycle genes.

### Example 11: Additional Candidate Bicycle Genes Can be Found in Many Other Species

To search for candidate bicycle genes in other species, the classifier was applied to all predicted genes from the genomes of twenty-two species spanning the Aphidoidea and species from the five most closely related families, the Phylloxeroidea, Coccoidea, Psylloidea, Aleyrodoidea, and Fulgoroidea (FIG. 1). Multiple candidate bicycle genes were identified in all Aphidoidea, Phylloxeroidea, and Coccoidea and the candidate bicycle genes identified by the classifier encompass 98.2-100% of the BLAST identified bicycle genes and 86.8-100% of the HMMER identified bicycle genes in all species with more than three bicycle genes. This concordance of methods supports the inference that 1) the classifier has identified bicycle homologs and 2) the classifier has higher power than sequence-based approaches to identify bicycle genes. As expected from the features of the classifier, the candidate bicycle genes in all species exhibited the unusual feature of being encoded by genes containing many micro-exons (FIG. 4).

While the classifier exhibits substantial power to detect highly divergent bicycle genes across species, the classifier might fail to identify bicycle genes with gene structures that are divergent from the bicycle genes originally identified in H. cornu that were used to train the classifier. In addition, the genomes of many species studied here are highly fragmented, and some true bicycle genes may have been annotated with a subset of their exons. Therefore, a second profile hmmer search was performed within each species starting with the classifier-identified bicycle genes in each species (last column in FIG. 1). This second hmmer search identified additional candidate bicycle genes in some species, suggesting that the classifier underestimates the number of bicycle genes in some species.

### Example 12: Candidate Bicycle Genes in H. cornu are Highly Expressed and Share Structural Homology

When the classifier was applied to all genes in the H. cornu genome, 653 candidate bicycle genes were identified, compared with the 476 bicycle genes previously described (Korgaonkar et al., 2021). Four originally defined bicycle genes were not detected and are thus false negatives. In addition, 181 newly detected bicycle genes were detected and are thus “false positives.” Since a probability cutoff was chosen that should report an approximately equal number of false negatives and false positives, it was hypothesized that the vast majority (about 181-4) of “false positives” are newly discovered bicycle homologs.

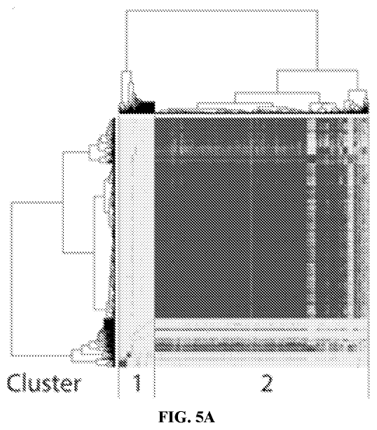

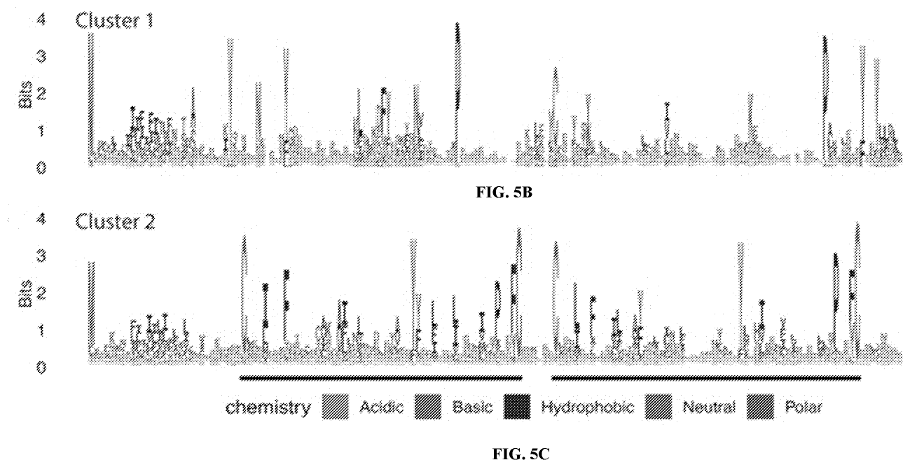

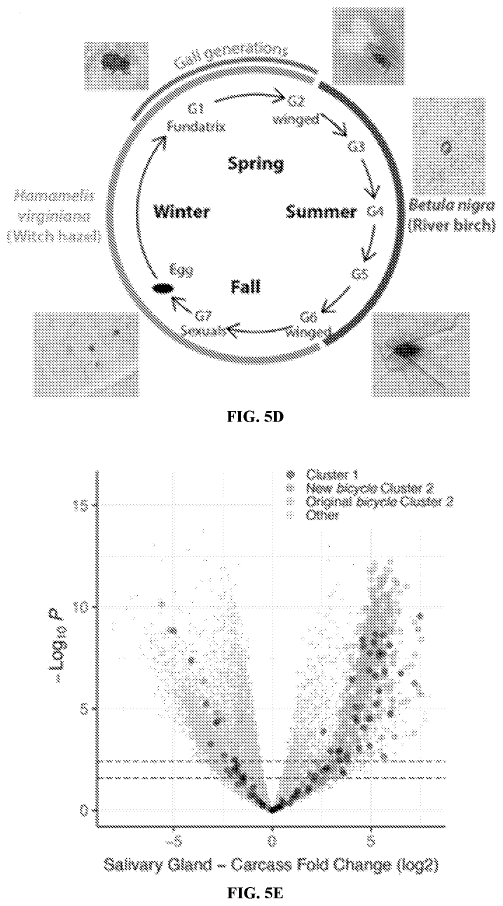

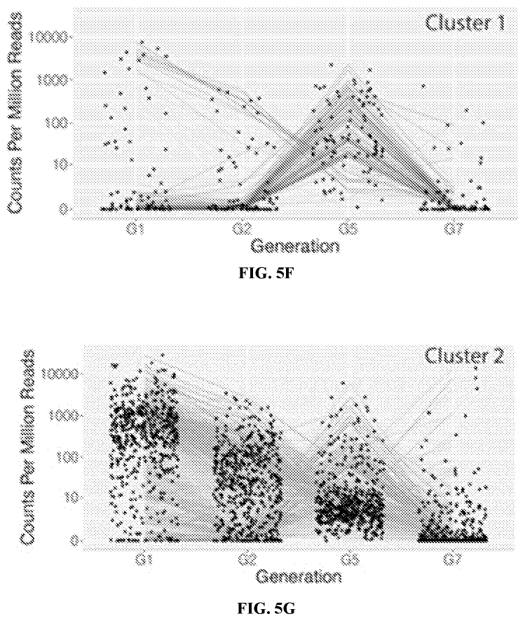

These newly discovered bicycle genes were clustered together with the original bicycle genes based on sequence similarity (FIG. 5A), and it was found that 563 represented a single group of related genes (Cluster 2, FIG. 5A) that all share sequence similarity with, and included, all but one of the originally defined 476 bicycle genes (FIG. 5C).

A second cluster of 94 genes was also identified that encode proteins that do not share strong sequence similarity with the originally defined Bicycle proteins and contain degenerated CYC motifs (Cluster 1, FIGS. 5A and 5B). Approximately 20 of these genes were strongly enriched in the salivary glands of gall-inducing foundresses, but many of these Cluster 1 genes were strongly enriched in salivary glands of other life stages, especially generations that feed on Betula nigra (River Birch) (FIG. 5D-5F). In addition, approximately 20 bicycle genes containing the CYC motif (Cluster 2) were weakly expressed in fundatrix salivary gland, but strongly expressed at other life stages (FIG. 5E-5G).

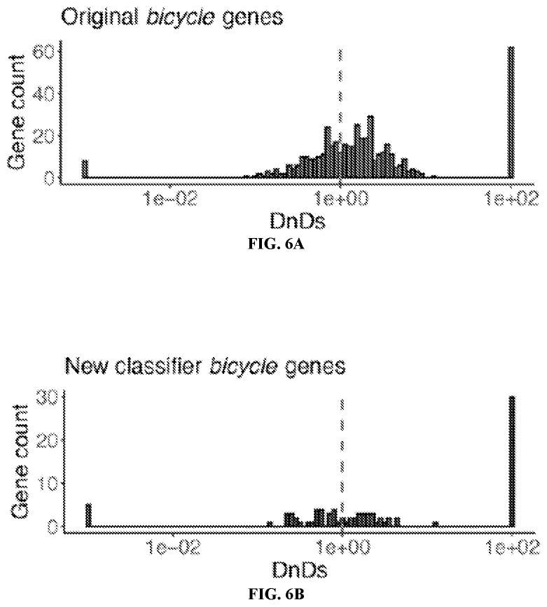

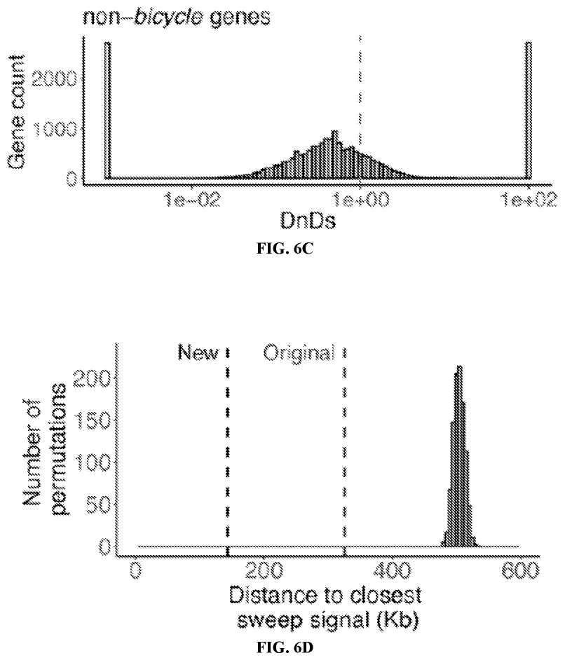

Like the original 476 bicycle genes, the new putative bicycle homologs likely experienced increased levels of positive selection. The new putative bicycle homologs displayed a similar pattern to the original bicycle genes of elevated ratios of non-synonymous (dN) to synonymous (dS) substitutions between H. cornu and its closely related sister species H. hamamelidis compared to the genomic background (FIG. 6A-6C). In addition, a genome-wide selection scan was performed using a site frequency spectrum-based composite likelihood ratio test that calculates the likelihood ratio of a sweep at a certain location in the genome to the neutral model. Signals of selective sweeps are enriched near both the original bicycle genes and the newly discovered putative bicycle homologs (FIG. 6D).

Since some of these new putative bicycle homologs shared little apparent sequence similarity with the originally defined bicycle genes, additional evidence for homology was sought. It was hypothesized that other details of gene structure not employed in the classifier, such as intron positions, might provide additional evidence of shared ancestry. Therefore, gene-structure-aware multiple sequence alignment was performed with the original bicycle genes and the new putative bicycle genes identified by the classifier (Gotoh, 2021).

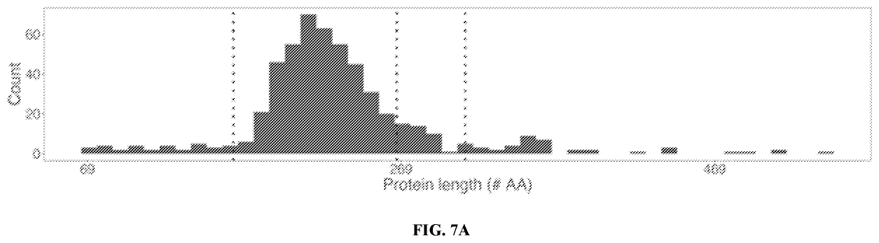

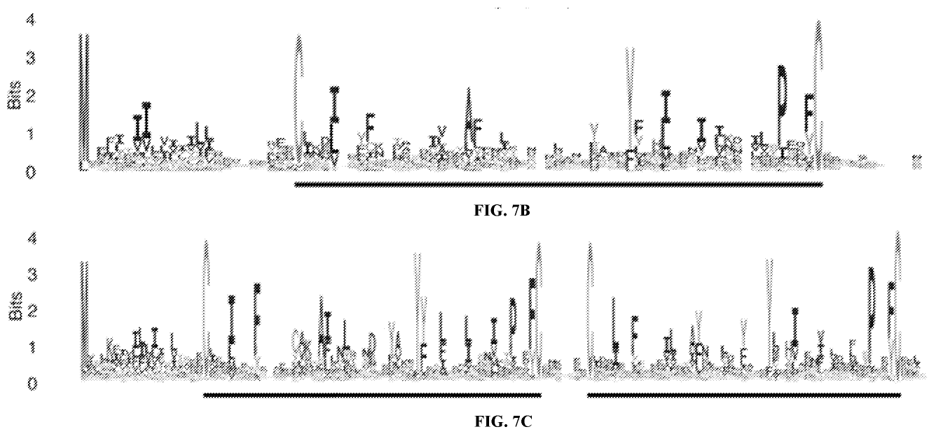

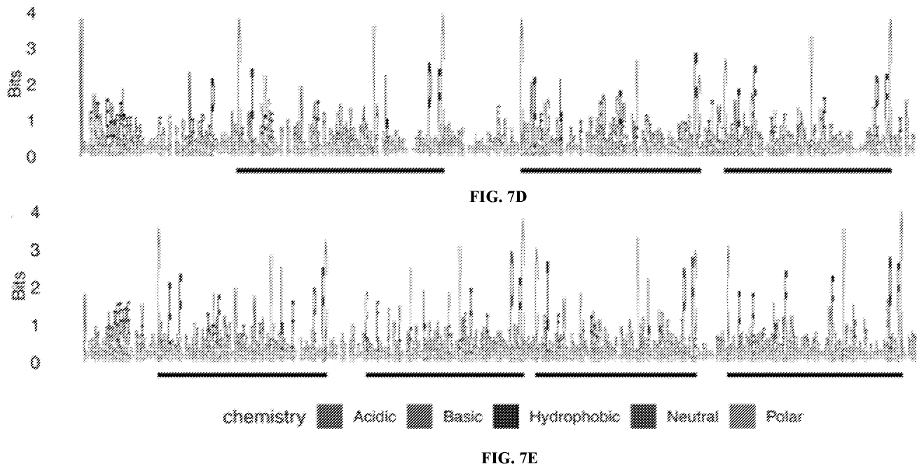

Initial attempts to align all 657 candidate proteins were uninformative and it was hypothesized that this was because these proteins display a large range of lengths and exon numbers (FIGS. 7A and 2E). Therefore Bicycle proteins were divided into four groups based on protein length (dotted lines in FIG. 7A). Logo plots of these four groups revealed a striking pattern, proteins contain one, two, three, or four CYC motifs (FIGS. 7B-7E), and therefore these were called unicycle, bicycle, tricycle, and tetracycle genes, respectively. This evolutionary comparison suggests that the CYC motif is a functional unit, which can be multimerized within individual proteins.

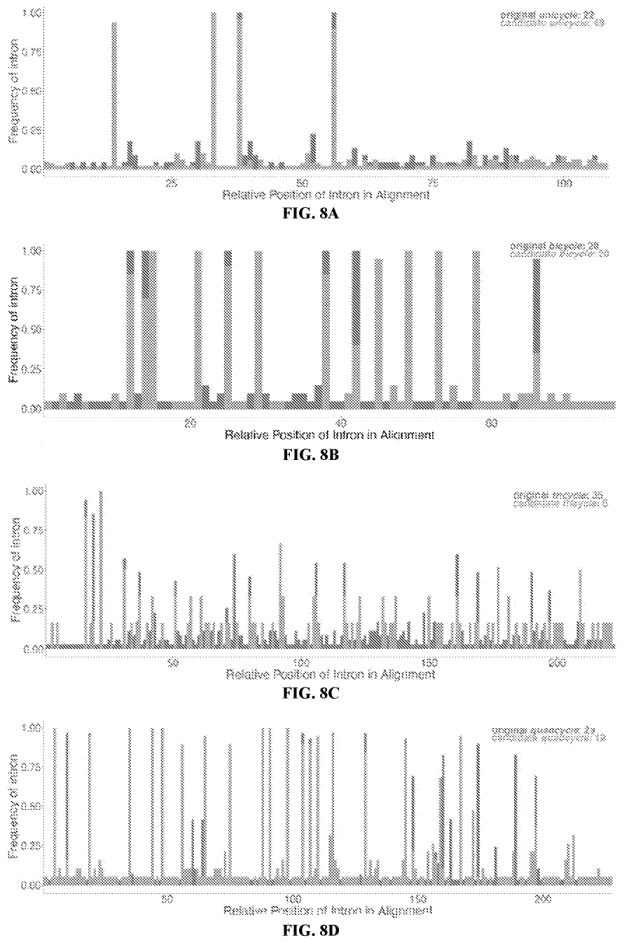

H. cornu bicycle homologs were divided into four length categories and performed gene-structure aware protein sequence alignments to search for shared intron position between the originally defined bicycle genes and the newly discovered bicycle putative homologs of Cluster 1. Many evolutionarily shared intron positions were identified between the two classes of putative homologs. (FIG. 8)

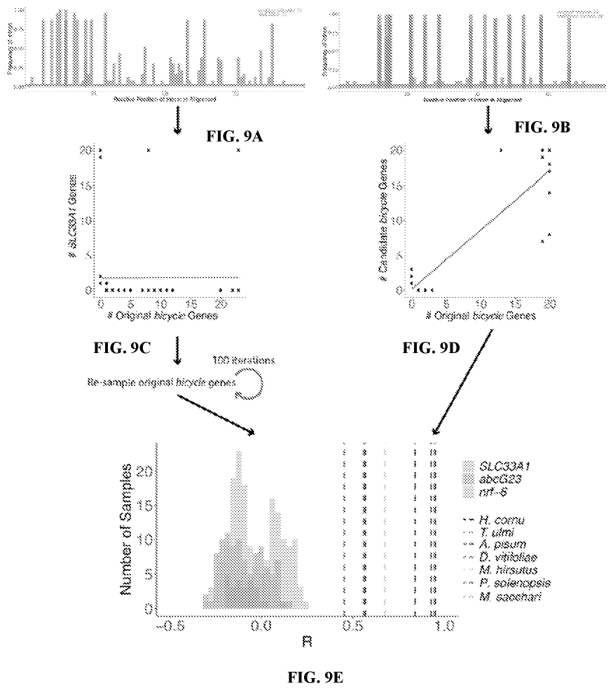

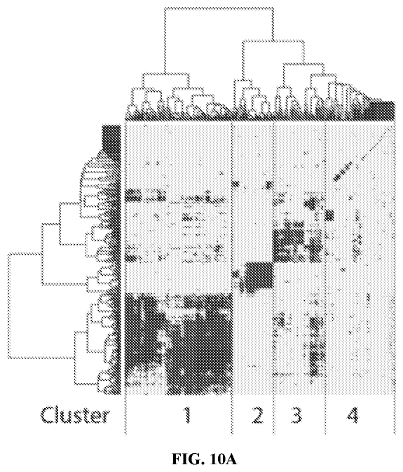

To determine whether more introns are shared between the original bicycle genes and newly identified putative bicycle genes than expected by chance—for example given an alignment of two groups of unrelated genes—a metric of intron concordance we developed and the null distribution was estimated by resampling intron concordance between bicycle genes and three unrelated gene families (FIG. 9). Intron concordance was measured as the correlation coefficient between the number of genes sharing an intron at each alignment position for one gene family versus the bicycle gene family (FIG. 9A-9D). Unrelated gene families were found to have intron positions distributed approximately randomly relative to bicycle genes with mean R close to 0 (FIGS. 9C and 9E). In contrast, newly discovered bicycle genes, both from H. cornu and from other species, displayed positive R much greater than 0 and significantly different from distributions of R values found for pairs of unrelated gene families (FIG. 9E). This analysis provides statistically significant support for the conclusion that newly discovered putative bicycle genes share introns in the same locations more often than expected by chance between unrelated gene families and thus that they are likely bicycle gene homologs.

These results provide evidence for several conclusions. First, measures of gene structure alone can reliably identify many, but perhaps not all, divergent bicycle genes. Second, some bicycle homologs have evolved highly divergent sequences, raising the possibility that highly divergent homologs may be present in other species but are undetectable with sequence-similarity search algorithms. Third, some bicycle homologs are highly expressed in salivary glands of non-gall forming life stages, suggesting that some of these proteins may contribute to the effector-protein repertoire outside of the context of gall development.

Given these observations, tests were conducted to determine whether the classifier could identify bicycle homologs in a divergent galling aphid species.

### Example 13: Many Candidate Gall Effector Genes in Tetraneura Nigriabdominalis are Highly Divergent Bicycle Genes

There are two major clades of gall forming aphids, the Hormaphididae, to which H. cornu belongs, and the Pemphigidae. These two aphid families are thought to have shared a common ancestor that induced galls. Therefore, to determine whether bicycle genes were present in the common ancestor of gall forming aphids, the genome of Tetraneura nigriabdominalis (FIG. 9A), a gall forming aphid belonging to the Pemphigidae, was assembled and annotated (Álvarez et al., 2013). The T. nigriabdominalis genome was annotated using mRNA sequencing reads generated from salivary glands and carcasses of the fundatrix (G1) and G2 generations. (FIG. 9B)

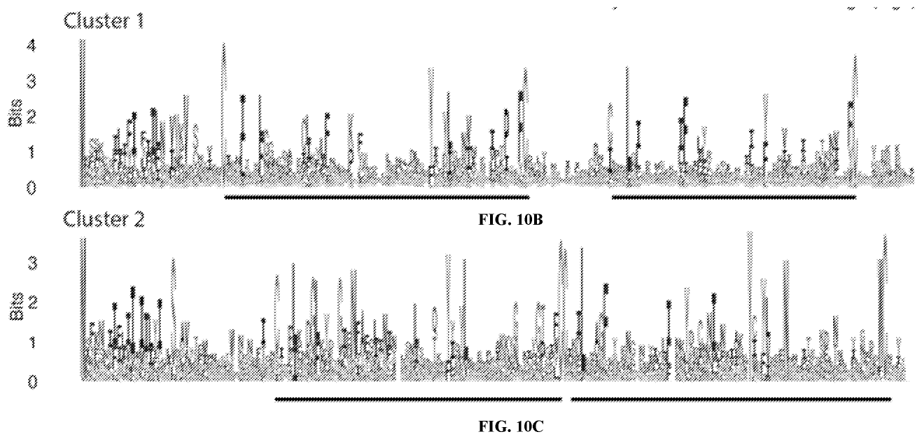

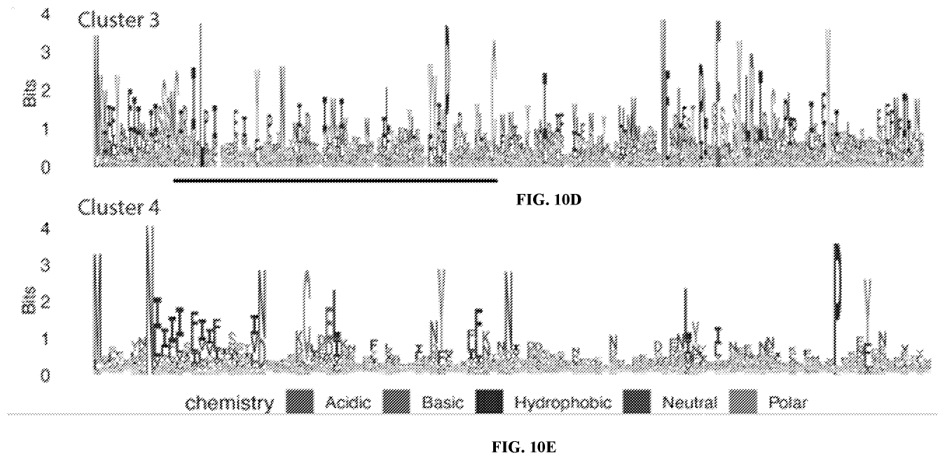

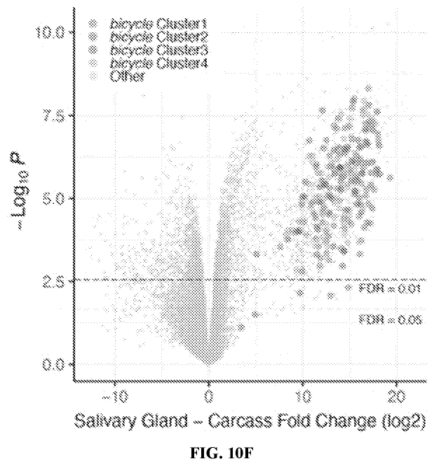

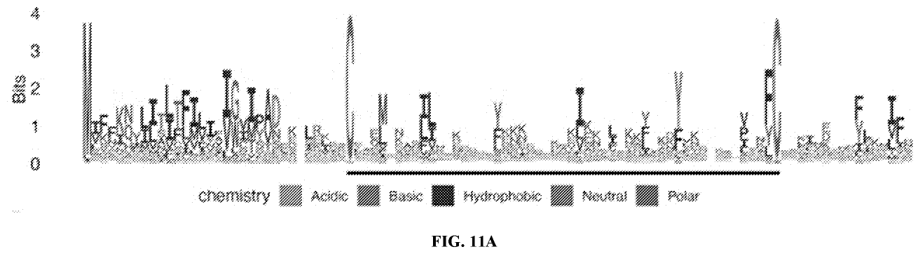

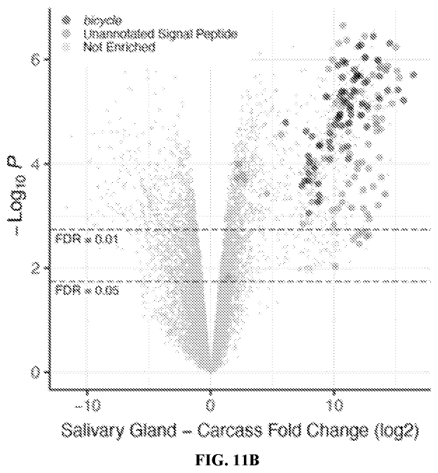

The H. cornu bicycle gene classifier was applied to predicted genes of the T. nigriabdominalis and 279 candidate bicycle homologs were identified (FIG. 1). In contrast, sequence-based search using BLAST and hmmer identified 1 and 53 candidate homologs at E-value<0.01, respectively. The 279 T. nigriabdominalis genes discovered by the gene-structure based classifier were clustered based on their predicted amino acid sequences (FIG. 10A) and three clusters were found to include proteins with clear evidence for CYC motifs. (FIG. 10B-10D). One cluster of 94 genes does not exhibit clear CYC motifs (FIG. 10E). The extreme sequence divergence of these genes within T. nigriabdominalis is supported by the fact that profile hmmer search recovered only 69 of these original 279 genes (FIG. 1).

To test whether the genes likely shared a common ancestor, gene-structure aware alignments of proteins were performed with and without CYC motifs and many shared introns were identified between putative homologs with and without CYC motifs (FIG. 9C) than expected by chance (FIG. 9E). Thus, the T. nigriabdominalis genome contains apparent bicycle genes that have become essentially unrecognizable as bicycle homolog at the sequence level.

To explore whether the T. nigriabdominalis bicycle genes, and especially the divergent putative bicycle homologs, might act as effector proteins, salivary glands were dissected from fundatrices and compared mRNA expression levels in salivary glands versus carcasses. Almost all of the bicycle homologs detected by the classifier are amongst the most strongly over-expressed genes in the fundatrix salivary glands (FIG. 10F), suggesting that they may contribute to the effector protein repertoire in T. nigriabdominalis.

The discovery of bicycle genes in T. nigriabdominalis using the gene-structure based classifier provides evidence that the classifier can identify putative bicycle homologs even when the genes cannot be identified using sequence-based homology methods and that bicycle genes were likely present in the common ancestor of gall-forming aphids. Next, tests were conducted to determine whether the classifier can detect putative bicycle homologs in an aphid species that does not induce galls.

### Example 14: The Pea Aphid (Acyrthosiphon pisum) Genome Includes Many Bicycle Homologs that are Candidate Effector Proteins

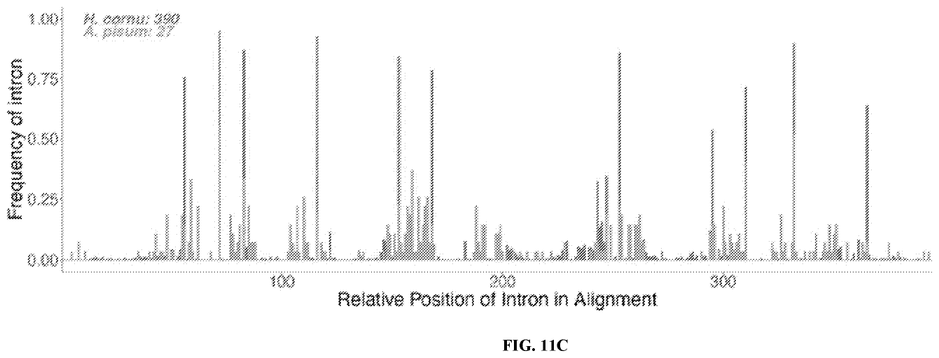

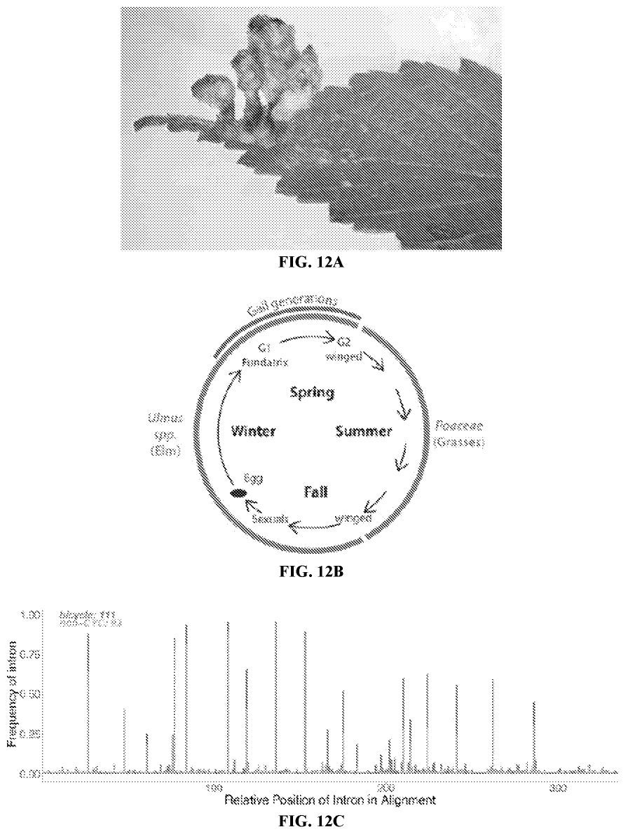

The gene-structure based classifier detected 121 putative bicycle homologs in the genome of Acyrthosiphon pisum, a species that does not induce galls. (Li et al., 2019; Richards et al., 2010). In contrast, sequence-based search using BLAST and hmmer identified 1 and 3 candidate hits at E-value<0.01, respectively. Multiple sequence alignment of the 121 A. pisum putative bicycle homologs revealed that most of these genes encode proteins with N-terminal secretion signal sequences and a single CYC motif (FIG. 11A). Gene structure aware alignment identified more shared introns between H. cornu bicycle genes and A. pisum putative bicycle homologs (FIG. 11C) than expected by chance (FIG. 9E), providing independent confirmation that these genes shared a common ancestor.

To explore whether these A. pisum bicycle homologs may contribute to the effector gene repertoire of A. pisum, RNA-seq of salivary glands and carcasses was performed. It was determined that 55% of the putative bicycle genes are among the most strongly overexpressed genes in salivary glands, along with a similar number of additional unannotated genes with signal peptides (FIG. 11B). In addition, 17 proteins encoded by genes with multiple microexons were identified by a previously-published proteomic study of proteins secreted by A. pisum into an artificial food medium (Dommel et al., 2020), and at least 13 of these proteins are bicycle homologs (Table 5). In summary, the A. pisum genome encodes approximately 121 bicycle homologs that may contribute to the A. pisum effector protein repertoire.

TABLE 5
Many microexon genes producing secreted putative effector proteins in
A. pisum detected by mass spectrometry of secreted proteins (Dommel et
al., 2020) are identified as bicycle genes by the classifier.

| Gene | | | Best hit | | | bicycle | |
|---|---|---|---|---|---|---|---|
| Name | Transcript | | Augustus | | SEQ | gene | |
| (Dommel | ID | Gene Locus | gene | Peptide sequence | ID | accordin | Notes |
| KQYla, b | NM_001162442, | LOC100158789 | g118916 | MIFFKQYSMMITFIVIAV | 1 | Yes | |
| | AK339882 | | | WVMPAITSE | | | |
| KQY2a, b | AK342599 & | LOC100302371 & | g2591 | MVFYKQYLLTITCIVITA | 2 | Yes | |
| | AK342927 | LOC100302371 | | WVIPTSA | | | |
| KQY3 | AK341661 | LOC100301916 | g48042 | MVFFKQYLITLTCIVISV | 3 | Yes | |
| | | | | WITPVNT | | | |
| KQY4a, b, | AK342948, | LOC100302370 | g139706 | MIFFKQYLIILTFIVIAV | 4 | Yes | |
| c, d | AK342242, | | | LVMPVTP | | | |
| | AK342690, | | | | | | |
| | AK342678 | | | | | | |
| KQY5a, b | AK342406, | LOC100302375, | g108486a | MVFFKQYLLTLTCIVIVV | 5 | Yes | |
| | AK342378 | LOC100302376 | | QVMPASA | | | |
| KQY6 | AK340126 | LOC100302481 | g119690 | MIFFKQYLIMLTFIIIAV | 6 | Yes | |
| | | | and g3762 | WVMPANT | | | |
| KQY7 | AK340563 | LOC100302485 | g103944 | MSFFKQYLTLTFIVISVW | 7 | Yes | |
| | | | | NMSEA | | | |
| KQY8 | AK342473 | LOC100302439 | g123349 | MVFFKQFLITLTVIIITE | 8 | ? | |
| | | | | A | | | |
| KQY9 | AK342683 | LOC100302403 | No close | MVFFKLYLLTLTCIVIAV | 9 | No | Closest |
| | | | matches | WVMPVSA | | | match, |
| | | | | | | | 76% |
| | | | | | | | identity |
| | | | | | | | to |
| KQY10 | AK342808 | LOC100302381 | g39312 | MFNVLIILSLISYTFEPS | 10 | Yes | |
| | | | | YTLYKFKMVFFKQDLLML | | | |
| | | | | TCITIAVWIMPPSASTN | | | |
| KQY11 | AK340121 | LOC100302480 | g144677 | MVFFRQFLITLSVILITE | 11 | No | |
| | | | | A | | | |
| KQYMp | XM_022322515 | LOC111039170 | g115805 | MHFFKHYLIVLTYIVISF | 12 | Yes | Myzus |
| | | | | WFMPSASL | | | persicae |
| KHI1a, b | AK339863, | LOC100159750 | g119277 | MFKHIIVLVLCFMAYFVG | 13 | Yes | 85% |
| | AK339862 | | | NLDA | | | identical |
| KHI2a, b, | AK341162, | LOC100302383 | g101192 | MDKHIIMLALCLMVYIIG | 14 | Yes | |
| c | AK341161, | | | NIDA | | | |
| | AK342769 | | | | | | |
| KHI3a, b | AK340197, | LOC100166702 | g134738 | MLKHIIVLALYLMAYIIG | 15 | Yes | |
| | AK342603 | | | NIDA | | | |
| KHI4 | AK341077 | LOC100570519 | g141770 | MLKHILLALCFMAYIIEN | 16 | Yes | |
| | | | | IG | | | |
| KHI5 | AK341390 | LOC100534636 | ? | MLKHILLALCFMAYIIEN | 17 | | g141770 |
| | | | | IGA | | | closest |
| | | | | | | | match, |
| | | | | | | | but no |
| | | | | | | | match |
| KHI6 | AK340760 | LOC100571631 | g121945 | MLKHIIVLVLCFMPYIIG | 18 | Yes | 94% |
| | | | | | | | identical |
| C002Ap | XM_001948323 | LOC100167863 | g50444 | MGSYKLYVAVMAIAIAVV | 19 | No | 2 exon |
| | | | | QEVRC | | | gene; not |
| | | | | | | | searched |

### Example 15: Bicycle Genes are Present in Genomes of Aphids, Phylloxerids, and Coccids

The presence of bicycle homologs in three divergent aphid species, H. cornu, T. nigriabdominalis and A. pisum implies that bicycle genes were present in the common ancestor of aphids, which lived approximately 280 MYA. To further explore the origins and evolution of bicycle genes, genomes and RNAseq data were downloaded for nine additional aphid species and ten outgroup species from NCBI. Predicted genes in all genomes were annotated using the same bioinformatic pipeline used previously to discover bicycle genes in other species, applied the bicycle gene classifier to all predicted genes, and manually annotated all putative bicycle homologs guided by RNA-seq data. One caveat of this analysis is that many of these genomes are fragmented into many contigs and genes bridging contigs are often misannotated or not annotated, resulting in possible under-counting of bicycle homologs.

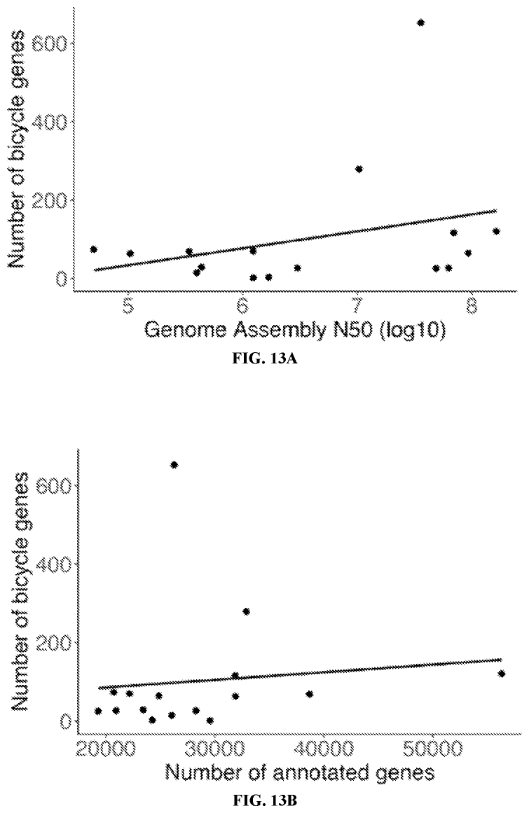

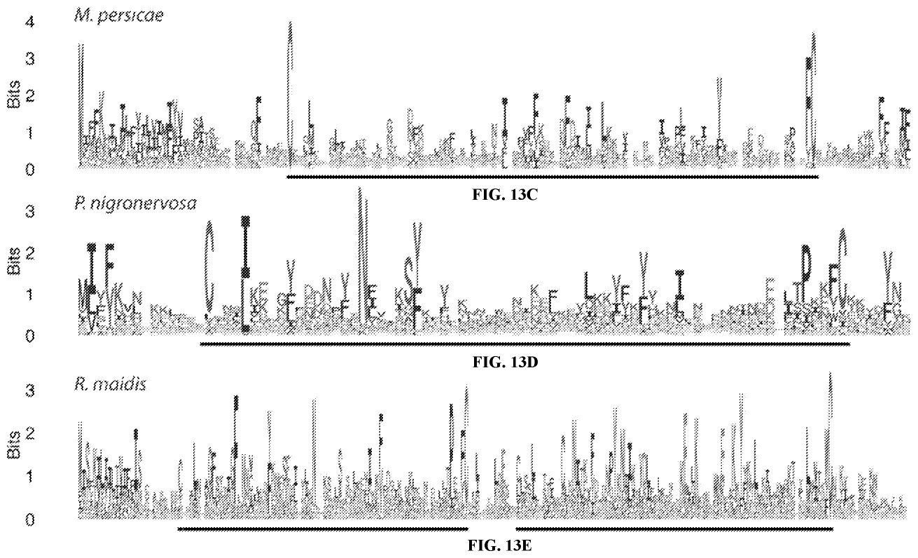

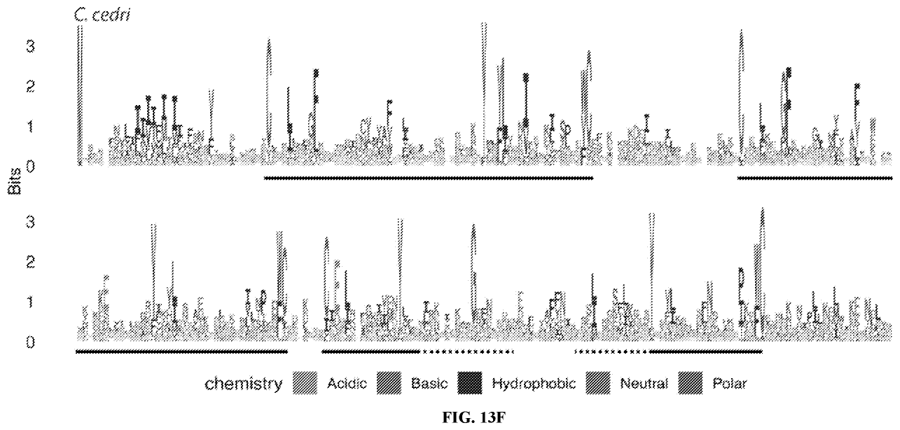

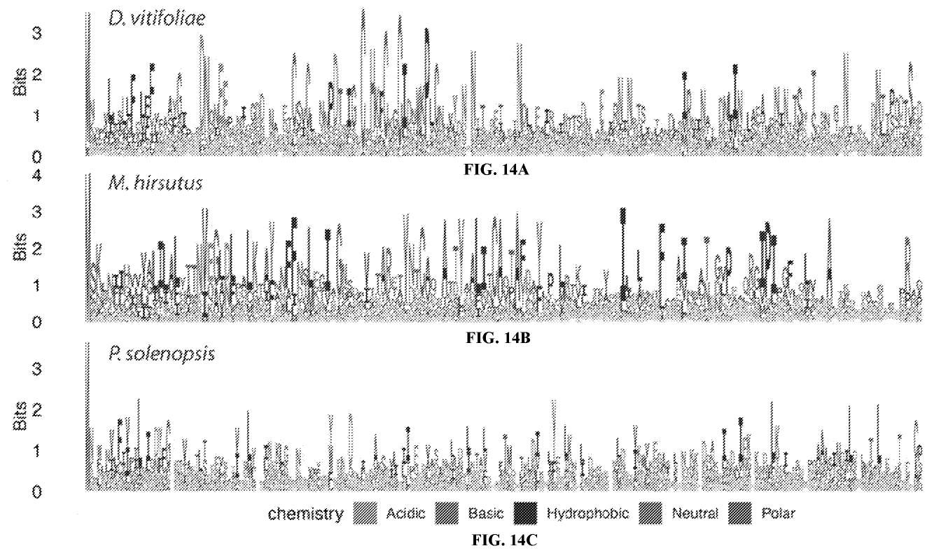

In addition, multiple putative bicycle homologs were detected near the ends of contigs, suggesting that parts of single bicycle homologs are present on multiple contigs, which would lead to over-counting bicycle gene homologs. While these problems with current annotations are expected to increase variance in estimates of the number of bicycle homologs, there was not a detection of a dependence of number of bicycle homologs detected on genome assembly quality (FIG. 13A) or on the number of genes annotated for each genome (FIG. 13B). In addition, chromosome-level genome assemblies were employed for two of the outgroup species where no predicted bicycle genes were found, P. venusta (Li et al., 2020) and T. vaporariorum (Xie et al., 2020), suggesting that the failure to identify bicycle genes in these species did not result from poor genome assemblies.

The phylogeny for these 22 species was estimated using whole-genome proteomic predictions (Emms, 2019), and this phylogeny is in general agreement with earlier studies based on a small number of genes (Johnson et al., 2018; Nováková et al., 2013; von Dohlen and N. A. Moran, 1995). Putative bicycle homologs were detected in all aphid species studied here, supporting the inference that bicycle genes were present in the common ancestor of aphids (FIG. 1). Extensive variation in the number of bicycle genes between species was found. In all aphid species with a sufficient number of bicycle homologs, multiple sequence alignment revealed the presence of CYC domains (FIG. 13C-13F). In most Aphidini species (e.g. M. persicae, A. pisum, P. nigronervosa), most homologs appear to be unicycle genes (FIG. 13C, 13D). In Cinara cedri, many homologs appear to be tetracycles (FIG. 13F).

Sixty-nine (69) putative bicycle homologs were found in Daktulosphaira vitifoliae indicating that bicycle genes were present in the common ancestor of the Aphidomorpha (Aphidoidea+Phylloxeridea). Seventy-four (74), 26, and 2 putative bicycle homologs were also found in the coccid species Maconellicoccus hirsutus, Phenacoccus solenopsis, and Ericerus pela, respectively. In M. hirsutus and E. pela profile hmmer search with the classifier identified genes identified 17 and 12 additional candidate bicycle genes, respectively.

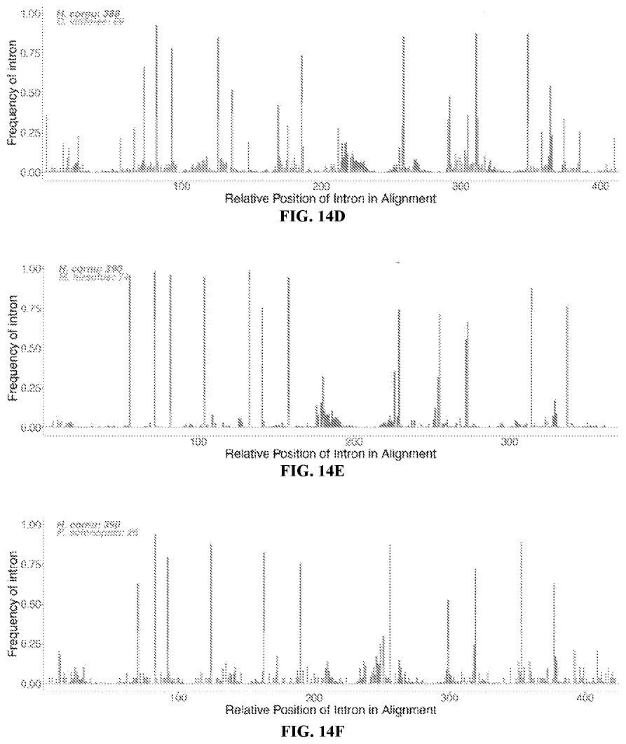

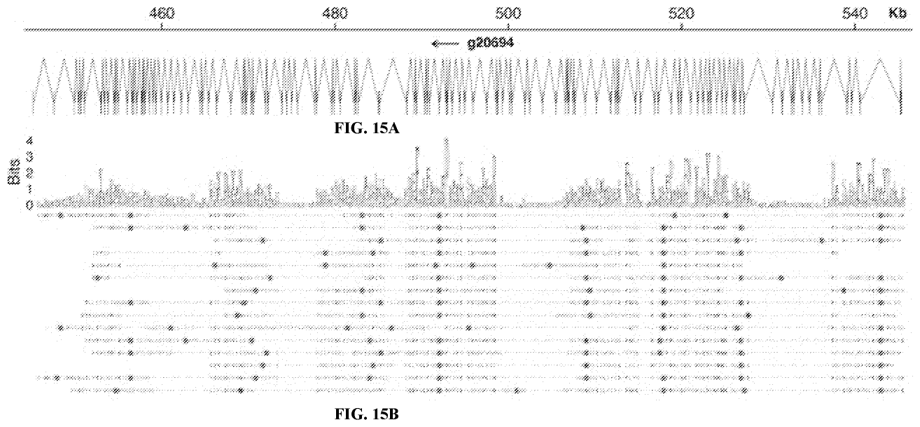

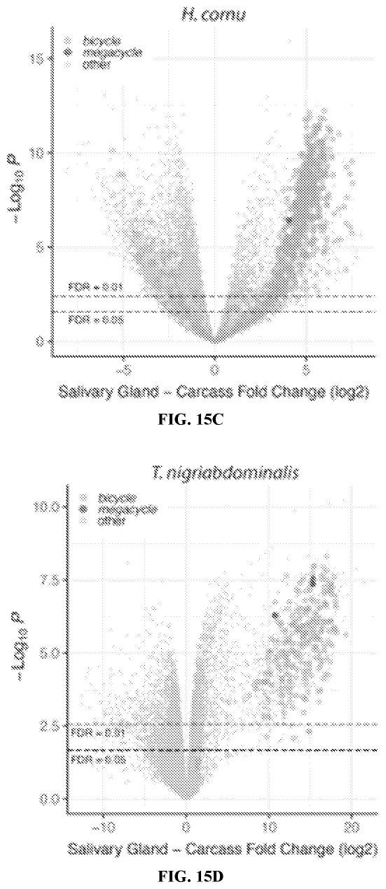

The putative bicycle homologs from these non-aphid species do not contain obvious CYC motifs (FIG. 13A-13C). However, gene structure aware alignment with the H. cornu bicycle genes revealed that the putative homologs from D. vitifoliae, M. hirsutus, and P. solenopis share more introns in the same locations with H. cornu bicycle genes (FIG. 14D-14F) than expected by chance (FIG. 14E), supporting the inference that these are bicycle homologs.

Bicycle homologs were not detected in any of the species sampled from the Psylloidea, Aleyrodoidea, or Fulgoroidea. These results indicate that bicycle genes were present in the common ancestor of coccids and aphids and may have evolved in the common ancestor of these lineages. However, for at least two reasons the possibility that bicycle genes originated earlier cannot be ruled out. First, extensive variation in the number of bicycle homologs were observed across lineages, so bicycle genes may have been lost in the specific outgroup species studied here. Second, ancestral bicycle genes may not display the specific gene structure detected by the classifier. Deeper taxonomic sampling and other homology search approaches may reveal an older origin of bicycle genes.

### Example 16: Aphid Genomes Contain a Conserved Megacycle Gene

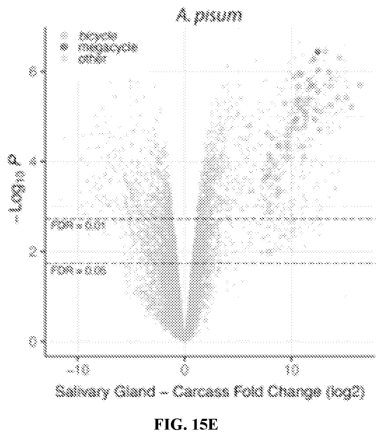

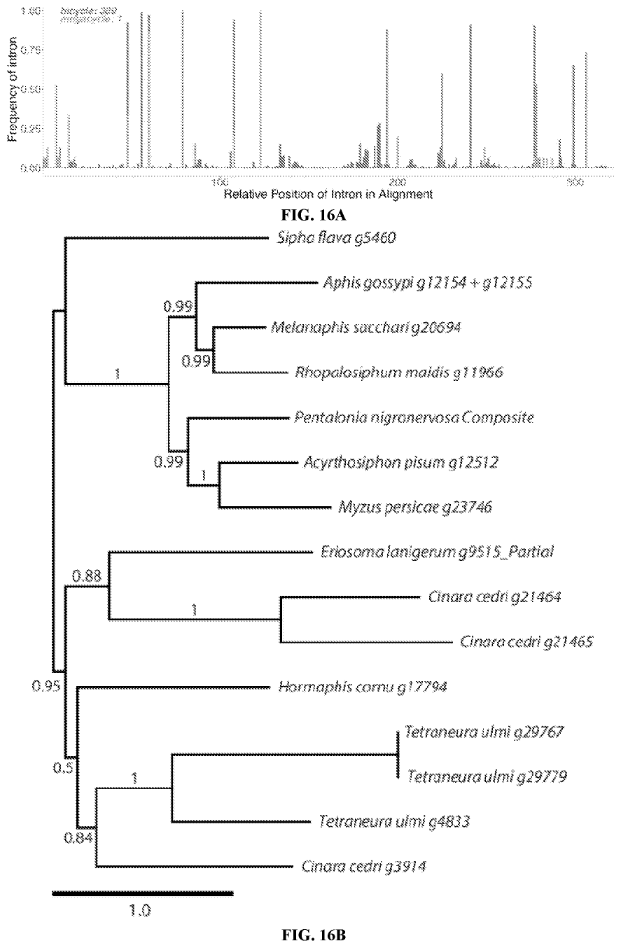

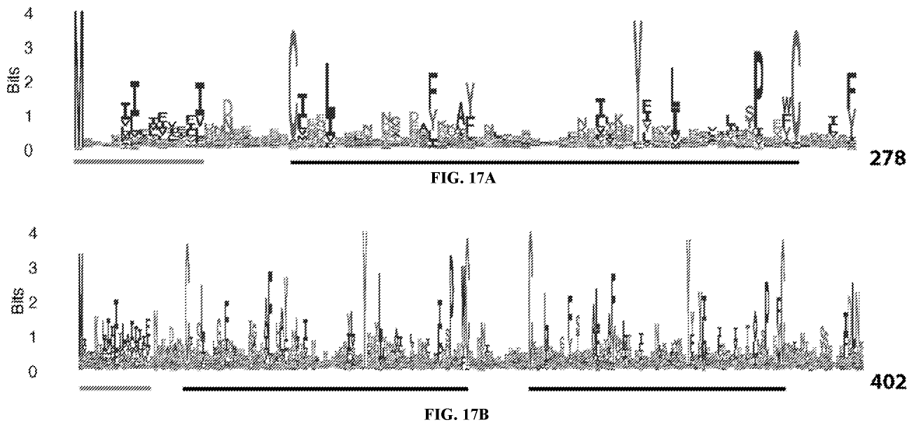

In 11 of the 12 aphid genomes that were studied, the classifier identified a single extremely large gene containing more than 100 microexons and encoding a protein of approximately 2000 amino acids as a candidate bicycle homolog (FIG. 15A). Since this gene was so large and most existing aphid genomes are incompletely assembled, many of these gene models were clearly incomplete. Therefore, de novo transcript assembly was performed for all species studied using Trinity (Grabherr et al., 2011), and consensus transcripts of these long candidate bicycle genes were assembled manually.

Since it was found that bicycle gene length is correlated with the number of encoded CYC motifs (FIG. 15A-15E), studies were conducted to determine whether these genes encoded proteins with multiple repeating units. Using Rapid Automatic Detection and Alignment of Repeats (Heger and Holm, 2000) via EMBL's European Bioinformatics Institute analysis tool (EMBL-EBI) (Madeira et al., 2019), these large proteins were found to contain N-terminal secretion signal and many repeating motifs and each motif is approximately the size of two CYC motifs. This structure was most obvious for the Melanaphis sacchari gene (FIG. 15A). Sequence-based alignment of 15 predicted repeats of the M. sacchari protein, which did not use information of intron positions, revealed multiple shared intron locations across repeats (FIG. 15B). Thus, this protein appears to have evolved through multiple internal tandem duplications. This large protein exhibited little obvious sequence similarity to bicycle genes, but gene-structure aware alignment revealed multiple apparently conserved intron positions shared between the internal duplications of the large gene from M. sacchari and H. cornu bicycle genes (FIG. 16A), suggesting that they shared a common ancestor.

These large genes appear to be divergent Bicycle proteins that have duplicated the core motif many times. Therefore, these are called megacycle genes. Compared to most bicycle genes, megacycle orthologs are relatively well conserved across aphids and a phylogenetic tree reconstructed from the inferred Megacycle proteins is similar to the aphid phylogeny estimated from the whole proteome (FIG. 163). Most aphid genomes that were examined contain a single copy of this gene; this gene was not detected in Diuraphis noxia, and three paralogs were found in both Cinara cedri and Tetraneura nigriabdominalis. No megacycle orthologs were found in any of the non-aphid species.

For the three species for which there was good salivary gland transcriptomic data, H. cornu, A. pisum and T. nigriabdominalis, the megacycle gene is strongly expressed in salivary glands (FIG. 15C-15E). Thus, megacycle genes may contribute to the effector protein repertoire in aphids. The relatively conserved lengths and sequences of megacycle homologs suggests that they may share a relatively conserved role across aphids.

### Example 17: Homology Independent of Sequence

Some gene features, such as the structure of the encoded protein or the intron-exon structure of the gene, can provide evidence for homology that is independent of sequence. (Bazan, 1991; Brown et al., 1995; Betts, 2001).

This source of information is becoming increasingly available with the recent availability of many well-assembled, annotated genomes. It is possible to conceptualize a model that jointly utilizes both sequence and exon-intron structure in homology search, but it was realized that bicycle genes are encoded with extremely unusual intron-exon structures and that a model incorporating only general features of exons may allow detection of distant homologs.

A linear classifier using information on gene length, exon sizes, exon numbers, and exon phases provided a highly accurate predictor of bicycle homologs that could not be identified using sequence similarity searches. This predictor allowed identification of bicycle homologs across aphids and in several outgroup taxa (FIG. 1). Homology was supported by post hoc observation of CYC motifs and shared introns across many homologs. Thus, sequence independent features provide substantial evidence of homology and integration of even a few sequence independent features into existing sequence-based search methods may substantially increase power to detect homology relationships between genes. All gene structure features that were used contributed to classifier performance, but no single feature was critically important for classifier performance. Therefore, in future work it would be ideal to incorporate multiple features of gene structure into homology search models.

For several reasons, the classifier may have underestimated the number of bicycle homologs in each species. First, this approach is sensitive to the quality of genome annotation, which is itself dependent on genome assembly quality. Most automatically annotated gene models of bicycle genes required manual re-annotation. Thus, additional bicycle genes in these genomes may have been overlooked because of genome fragmentation and inaccurate automated gene annotation. Second, bicycle gene annotation in most species is likely hampered especially by the lack of salivary gland RNA-seq samples. Considerable variation in bicycle gene expression levels was observed among samples, and deep sequencing of diverse samples is likely required to provide sufficient RNAseq evidence to build accurate bicycle gene models. Finally, it is possible that some bicycle homologs have evolved both divergent sequences and novel gene structures, preventing identification with any existing method.

### Example 17: Bicycle Genes are not Strictly Associated with Gall Formation

Bicycle genes were detected originally as putatively secreted proteins strongly expressed in the salivary glands of the gall-forming generation of the aphid H. cornu and variation in one bicycle gene is genetically associated with gall color (Korgaonkar et al., 2021). These observations led to the hypothesis that many bicycle genes participate in gall development. The observations provide a revised assessment of potential Bicycle protein functions.

First, many bicycle genes are most strongly expressed in salivary glands of generations of H. cornu that do not induce galls (FIG. 5F-5G). Second, two of the aphids studied here, H. cornu and T. nigriabdominalis induce complex galls and their genomes include many more bicycle genes than other species that were examined, but the genomes of two other gall-forming species, E. lanigerum and D. vitifoliae have only 27 and 69 bicycle genes, respectively. In addition, the genomes of some non-galling aphids have many bicycle genes, such as Myzus persicae and A. pisum, with 117 and 121, respectively. Thus, across species there does not appear to be a strong correlation between the number of bicycle genes and the gall-forming habit.

Given these new observations, if Bicycle proteins have conserved molecular functions, then they likely have different targets in different plant species. In some cases, the targets may induce patterned cell proliferation, resulting in galls, and in others the targets may alter plant physiology in more subtle ways to confer benefits on aphids.

None of the three coccid species that were studied induce galls, However, many coccids induce depressions, swellings, or other changes in plant organs (Beardsley and Gonzalez, 1975), including the induction of elaborate galls (Cook and Gullan, 2004). Parr (1940) reported a remarkable series of experiments that appear to demonstrate that extracts of salivary glands of a coccid are sufficient to induce pit galls on oak trees. It may be worth exploring the hypothesis that bicycle genes contribute to the effector gene repertoire of scale insects and especially of the gall-forming species.

### Example 18: New Opportunities Provided by the Presence of Bicycle Genes in Many Species

One of the challenges with studying the function of bicycle genes is that initially they were thought to be restricted to the non-model gall-forming insect H. cornu. H. cornu displays a complex life cycle, exhibiting many polyphenic forms and migration between two trees every year (Korgaonkar et al., 2021), and prospects for laboratory rearing and subsequent experimental manipulations of this species were poor.

The discovery of bicycle genes in other insects opens up the possibility that the function of bicycle genes can be studied in species more amenable to laboratory manipulation. For example, RNAi allows gene knockdown in M. persicae (Pitino et al., 2011; Coleman et al., 2015; Mathers et al., 2017; Niu et al., 2019; Chen et al., 2020; Gao et al., 2021) and A. pisum (Han et al., 2006; Mutti et al., 2006, 2008; Shakesby et al., 2009; Will and Vilcinskas, 2015; Vellichirammal et al., 2017; Niu et al., 2019) and CRISPR-Cas9 mutagenesis allows gene knockout in A. pisum (Le Trionnaire et al., 2019). Thus, the discovery of many bicycle genes in these species presents new experimental opportunities to uncover the organismal and molecular functions of bicycle genes.

### Example 19: Application of Method for Identifying Bicycle Genes

The method for detecting bicycle genes, as disclosed herein, was applied to high-quality genome and transcriptome data that was published by Wei et al. in connection with their publication entitled “Chromosome-level genome assembly for the homed-gall aphid provides insight into interactions between gall-making insect and its host plant.” (Wei et al., 2022).

Gene annotation was conducted by Wei et al. to screen the S. chinensis genome against available databases, and more than 79 million bp repetitive sequences were obtained in the S. chinensis genome (Wei, et al. 2022, 2.5 Gene annotation and 3.3 Genome annotation). Among the analyses conducted, there was a search for Bicycle proteins; however, Wei et al. “did not identify any BICYCLE protein in the salivary glands of S. chinensis.” (Wei, et al. 2022, 3.5 Salivary protein-encoded genes and other gall formation associated genes).

Contrary to this finding, when the method disclosed herein for detecting bicycle genes was applied to the genome and transcriptome data published by Wei et al., hundreds of bicycle genes were identified. The identification of these genes as bicycle genes by the method of the presently-disclosed subject matter was validated as follows.

Briefly, to determine whether the predicted bicycle genes are true bicycle genes, the following four validation steps were applied.

First, it was determined whether the genes encode proteins with N-terminal secretion signal sequence. Proteins must be secreted from the insect salivary glands to allow them to be introduced into the plant. The N-terminal secretion signal allows this secretion to occur.

Second, it was determined whether the sequence alignment yields a conserved CYC motif. The bicycle genes were originally identified based on the presence of a conserved cysteine-tyrosine-cysteine (CYC) motif. In most cases, bicycle genes identified by the classifier (the method disclosed herein) contain this motif, either in full or degenerate form. In some cases, candidate bicycle genes do not contain a CYC motif, but in all such cases of bicycle genes identified by the classifier, when these genes are exampled closely, they are found to share extensive sequence similarity to bicycle genes that do contain the CYC motif.

Third, it was determined whether the sequence alignment that accounts for intron positions reveals more conserved intron positions than expected by chance. As disclosed herein, a large excess of shared intron positions have been identified amongst bicycle genes. This has turned out to be a very reliable criterion for identifying bicycle genes.

Fourth, it was determined whether the genes are over-expressed in relevant tissue. This step need not be performed in addition to the first three validation steps; indeed, it cannot be performed for all datasets, because sometimes the salivary glands of the gall-inducing stage are not available. However, in all examined cases, if bicycle genes are expressed at all, then they are expressed specifically in the salivary glands. In aphids making galls, most bicycle genes are expressed during the gall-making life stages.

The identification of the hundreds of bicycle genes, when applying the method disclosed herein to the genome and transcriptome data published by Wei et al., was confirmed using these four validation steps.

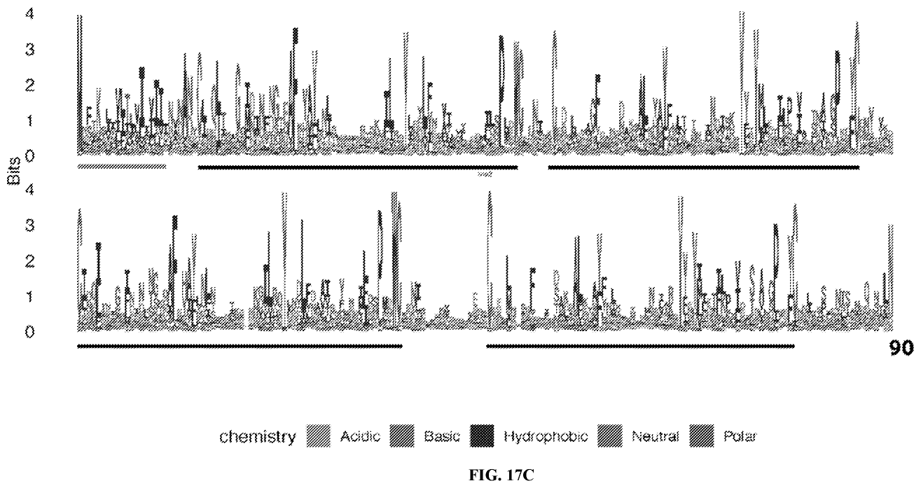

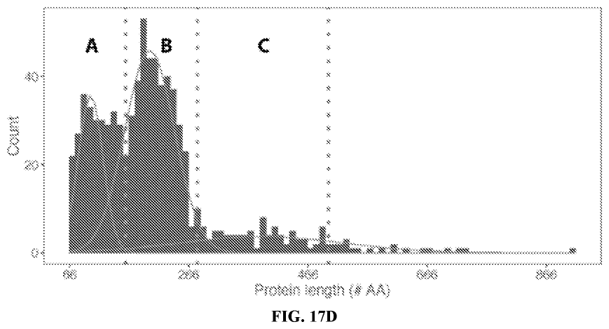

Using the classifier (method disclosed herein), 770 bicycle genes were predicted. With reference to FIG. 17A-17D, these predicted bicycle genes were divided into three groups based on their length (FIG. 17D—Group A including 278 predicted bicycle genes, Group B including 402, and Group C including 90). Sequence Logos for each size class were obtained (Group A —FIG. 17A, Group B—FIG. 17B, Group C—FIG. 17C). With regard to the first validation step, all predicted bicycle proteins contain N-terminal secretion signals (FIG. 17A-17C). With regard to the second validation step, all size classes show clear evidence for one (FIG. 17A) or multiple (FIGS. 17B and 17C) CYC motifs (sequences above black lines).

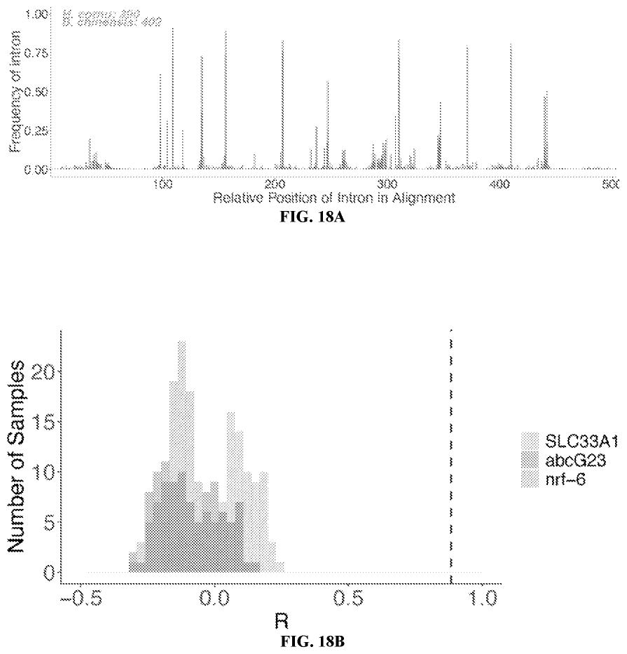

Turning now to FIGS. 18A and 18B, sequences were aligned with an intron-aware alignment method and shared intron positions were identified (FIG. 18A). The correlation coefficient (R) between the number of introns from each species at each position is shown as the dashed line in FIG. 18B. The distribution of correlation coefficients for control comparisons of unrelated genes are provided (FIG. 18B). The distribution of correlation coefficients for control comparisons of unrelated genes are also provided (FIG. 18B). The correlation coefficient between predicted bicycle genes in S. chinensis and bicycle genes from H. cornu lies far outside the distributions of control genes, providing strong evidence that the predicted bicycle genes in S. chinensis are true bicycle genes. Thus, with regard to the third validation step, the predicted bicycle genes from S. chinensis share more intron locations with bicycle genes of H. cornu than expected by chance.

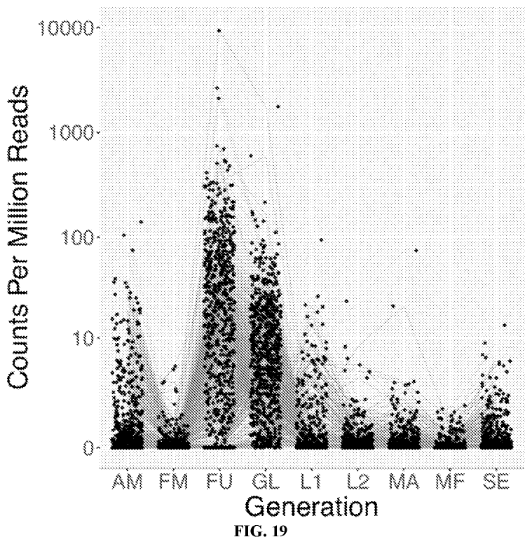

With reference to FIG. 19, plots are provided showing expression levels (in counts per million reads) of predicted bicycle genes from whole-animal RNA extracts of individuals from the following generations: AM—Autumn Migrant, FM—Sexual Female, FU Generation 1—Fundatrix, GL—Generation 2—Fundatriginae, L1—Nymph, L2—Nymph, MA—Sexual Male, MF—Pregnant Female, and SE—Spring Migrant. Bicycle genes are most highly expressed in the aphids that induce and live in galls (FU and GL), as expected of bicycle genes. Accordingly, with reference to the fourth validation step, expression of bicycle genes is biased toward the gall-forming generations.

Using the four validation steps, it was confirmed that the hundreds of bicycle genes that were identified, when applying the method disclosed herein to the genome and transcriptome data published by Wei et al., are true bicycle genes.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Aguiar ERGR, Olmo R P, Paro S, Ferreira F V, de Faria I J da S, Todjro Y M H, Lobo F P, Kroon E G, Meignin C, Gatherer D, Imler J-L, Marques J T. 2015. Sequence-independent characterization of viruses based on the pattern of viral small RNAs produced by the host. Nucleic Acids Research 43:6191-6206. doi:10.1093/nar/gkv587
2. Altschul S. 1997. Gapped BLAST and PSI-BLAST: a new generation of protein database search programs. Nucleic Acids Research 25:3389-3402. doi:10.1093/nar/25.17.3389
3. Altschul S F, Gish W, Miller W, Myers E W, Lipman D J. 1990. Basic local alignment search tool. Journal of Molecular Biology 215:403-410. doi:10.1016/S0022-2836(05)80360-2.
4. Álvarez R A, González-Sierra S, Candelas A, Martinez J-J I. 2013. Histological study of galls induced by aphids on leaves of Ulmus minor: Tetraneura ulmi induces globose galls and Eriosoma ulmi induces pseudogalls. Arthropod-Plant Interactions 7:643-650. doi:10.1007/si 1829-013-9278-8
5. Baek M, DiMaio F, Anishchenko I, Dauparas J, Ovchinnikov S, Lee G R, Wang J, Cong Q, Kinch L N, Schaeffer R D, Millán C, Park H, Adams C, Glassman C R, DeGiovanni A, Pereira J H, Rodrigues A V, Dijk A A van, Ebrecht A C, Opperman D J, Sagmeister T, Buhlheller C, Pavkov-Keller T, Rathinaswamy M K, Dalwadi U, Yip C K, Burke J E, Garcia K C, Grishin N V, Adams P D, Read R J, Baker D. 2021. Accurate prediction of protein structures and interactions using a three-track neural network. Science. doi:10.1126/science.abj8754
6. Bazan J F. 1991. Neuropoietic cytokines in the hematopoietic fold. Neuron 7:197-208
7. Beardsley J W, Gonzalez R H. 1975. The biology and ecology of armored scales. Annu Rev Entomol. 20:47-73
8. Betancourt A J, Presgraves D C. 2002. Linkage limits the power of natural selection in Drosophila. Proceedings of the National Academy of Sciences of the United States of America 99:13616-13620. doi:10.1073/pnas.212277199
9. Betts M J. 2001. Exon structure conservation despite low sequence similarity: a relic of dramatic events in evolution? EMBO J 20:5354-5360.
10. Birky C W, Walsh J B. 1988. Effects of linkage on rates of molecular evolution. Proceedings of the National Academy of Sciences of the United States of America 85:6414-6418. doi:10.1073/pnas.85.17.6414
11. Brown N P, Whittaker A J, Newell W R, Rawlings C J, Beck S. 1995. Identification and analysis of multigene families by comparison of exon fingerprints. J Mol Biol. 249:342-359.
12. Burstein M, Wool D, Eshel A. 1994. Sink strength and clone size of sympatric, gall-forming aphids. European Journal of Entomology 91:57-61.
13. Camacho C, Coulouris G, Avagyan V, Ma N, Papadopoulos J, Bealer K, Madden T L. 2009. BLAST+: Architecture and applications. BMC Bioinformatics 10:1-9. doi:10.1186/1471-2105-10-421
14. Chen Y, et al. 2020. An aphid RNA transcript migrates systemically within plants and is a virulence factor. Proc Natl Acad Sci USA 117:12763-12771.
15. Coleman A D, Wouters R H M, Mugford S T, Hogenhout S A. 2015. Persistence and transgenerational effect of plant-mediated RNAi in aphids. J Exp Botany. 66:541-548.
16. Cook L g., Gullan P j. 2004. The gall-inducing habit has evolved multiple times among the eriococcid scale insects (Stemorrhyncha: Coccoidea Eriococcidae). Biological Journal of the Linnean Society 83:441-452. doi:10.1111/j.1095-8312.2004.00396.x
17. Dixon A F G. 1978. Biology of aphids, Studies in Biology. London: Edward Arnold.
18. Dommel M, Oh J, Huguet-Tapia J C, Guy E, Boulain H, Sugio A, Murugan M, Legeai F, Heck M, Smith C M, White F F. 2020. Big Genes, Small Effectors: Pea Aphid Cassette Effector Families Composed From Miniature Exons. Front Plant Sci 11. doi:10.3389/fpls.2020.01230
19. Dunn N A, Unni D R, Diesh C, Munoz-Torres M, Harris N L, Yao E, Rasche H, Holmes I H, Elsik C G, Lewis S E. 2019. Apollo: Democratizing genome annotation. PLoS Comput Biol 15:e1006790. doi:10.1371/joumal.pcbi.1006790
20. Eddy S R. 2011. Accelerated profile HMM searches. PLoS Computational Biology 7. doi:10.1371/joumal.pcbi.1002195
21. Eizaguirre C, Lenz T L, Kalbe M, Milinski M. 2012. Rapid and adaptive evolution of MHC genes under parasite selection in experimental vertebrate populations. Nature Communications 3:1-6. doi:10.1038/ncomms1632
22. Elzinga D A, Jander G. 2013. The role of protein effectors in plant-aphid interactions. Current Opinion in Plant Biology 16:451-456. doi:10.1016/j.pbi.2013.06.018
23. Emms D M. 2019. OrthoFinder: phylogenetic orthology inference for comparative genomics 14.
24. Emms D M, Kelly S. 2018. STAG: Species Tree Inference from All Genes. bioRxiv 1-29. doi:https://doi.org/10.1101/267914
25. Gao Y, et al. 2021. The gustavus gene can regulate the fecundity of the green peach aphid, Myzus persicae (Sulzer). Front Physiol. 11:596392.
26. Giron D, Huguet E, Stone G N, Body M J A. 2016. Insect-induced effects on plants and possible effectors used by galling and leaf-mining insects to manipulate their host-plant Journal of Insect Physiology 84:70-89. doi:10.1016/j.jinsphys.2015.12.009
27. Gotoh O. 2021. Cooperation of Spaln and Prm5 for Construction of Gene-Structure-Aware Multiple Sequence Alignment In: Katoh K, editor. Multiple Sequence Alignment, Methods in Molecular Biology. New York, NY: Springer U S. pp. 71-88. doi:10.1007/978-1-0716-1036-7_5
28. Grabherr M G, Haas B J, Yassour M, Levin J Z, Thompson D A, Amit I, Adiconis X, Fan L, Raychowdhury R, Zeng Q, Chen Z, Mauceli E, Hacohen N, Gnirke A, Rhind N, di Palma F, Birren B W, Nusbaum C, Lindblad-Toh K, Friedman N, Regev A. 2011. Full-length transcriptome assembly from RNA-Seq data without a reference genome. Nat Biotechnol 29:644-652. doi:10.1038/nbt.1883
29. Han J, et al. 2006. Molecular basis for the recognition of primary microRNAs by the Drosha-DGCR8 complex. Cell 125:887-901.
30. Hebert P D N, et al. 2016. Counting animal species with DNA barcodes: Canadian insects. Phil Trans R Soc B. 371:20150333.
31. Heger A, Holm L. 2000. Rapid automatic detection and alignment of repeats in protein sequences. Proteins: Structure, Function, and Bioinformatics 41:224-237. doi:10.1002/1097-0134(20001101)41:2<224::AID-PROT70>3.0.CO;2-Z
32. Hoff K J, Lomsadze A, Borodovsky M, Stanke M. 2019. Whole-genome annotation with BRAKER, Methods in Molecular Biology. doi:10.1007/978-1-4939-9173-0_5
33. Hogenhout S A, Bos J I B. 2011. Effector proteins that modulate plant-insect interactions. Current Opinion in Plant Biology 14:422-428. doi:10.1016/j.pbi.2011.05.003
34. Johnson K P, Dietrich C H, Friedrich F, Beutel R G, Wipfler B, Peters R S, Allen J M, Petersen M, Donath A, Walden K K O, Kozlov A M, Podsiadlowski L, Mayer C, Meusemann K, Vasilikopoulos A, Waterhouse R M, Cameron S L, Weirauch C, Swanson D R, Percy D M, Hardy N B, Terry I, Liu S, Zhou X, Misof B, Robertson H M, Yoshizawa K. 2018. Phylogenomics and the evolution of hemipteroid insects. Proceedings of the National Academy of Sciences 201815820-201815820. doi:10.1073/pnas.1815820115
35. Johnson L S, Eddy S R, Portugaly E. 2010. Hidden Markov model speed heuristic and iterative HMM search procedure. BMC Bioinformatics 11. doi:10.1186/1471-2105-11-431
36. Jumper J, Evans R, Pritzel A, Green T, Figumov M, Ronneberger O, Tunyasuvunakool K, Bates R, Židek A, Potapenko A, Bridgland A, Meyer C, Kohl S A A, Ballard A J, Cowie A, Romera-Paredes B, Nikolov S, Jain R, Adler J, Back T, Petersen S, Reiman D, Clancy E, Zielinski M, Steinegger M, Pacholska M, Berghammer T, Bodenstein S, Silver D, Vinyals O, Senior A W, Kavukcuoglu K, Kohli P, Hassabis D. 2021. Highly accurate protein structure prediction with AlphaFold. Nature 1-11. doi:10.1038/s41586-021-03819-2
37. Korgaonkar A, Han C, Lemire A L, Siwanowicz I, Bennouna D, Kopec R E, Andolfatto P, Shigenobu S, Stem D L. 2021. A novel family of secreted insect proteins linked to plant gall development. Current Biology 31:1836-1849.e12. doi:10.1016/j.cub.2021.01.104
38. Lee W, Akimoto S-I. 2015. Development of new barcoding loci in gall-forming aphids (Eriosomatinae: Eriosomatini): comparing three mitochondrial genes, ATP6, ATP8, and COI. J Asia Pac Entomol. 18:267-275.
39. Le Trionnaire G, et al. 2019. An integrated protocol for targeted mutagenesis with CRISPR-Cas9 system in the pea aphid. Insect Biochem Mol Biol. 110:34-44.
40. Li Y, Park H, Smith T E, Moran N A, Singh N. 2019. Gene Family Evolution in the Pea Aphid Based on Chromosome-level Genome Assembly. Molecular Biology and Evolution 36:2143-2156. doi:10.1093/molbev/mszl38
41. Li Y, Zhang B, Moran N A. 2020. The Aphid X Chromosome Is a Dangerous Place for Functionally Important Genes: Diverse Evolution of Hemipteran Genomes Based on Chromosome-Level Assemblies. Molecular Biology and Evolution 37:2357-2368. doi:10.1093/molbev/msaa095
42. Loewenstein Y, Raimondo D, Redfem O C, Watson J, Frishman D, Linial M, Orengo C, Thornton J, Tramontano A. 2009. Protein function annotation by homology-based inference. Genome Biol 10:207. doi:10.1186/gb-2009-10-2-207
43. Madeira F, Park Y mi, Lee J, Buso N, Gur T, Madhusoodanan N, Basutkar P, Tivey A R N, Potter S C, Finn R D, Lopez R. 2019. The EMBL-EBI search and sequence analysis tools APIs in 2019. Nucleic Acids Research 47:W636-W641. doi:10.1093/nar/gkz268
44. Mani M S. 1964. Ecology of Plant Galls. The Hague, The Netherlands: Springer-Science+Business Media, B.V. doi:10.2307/3756723
45. Mathers T C, et al. 2017. Rapid transcriptional plasticity of duplicated gene clusters enables a clonally reproducing aphid to colonise diverse plant species. Genome Biol. 18:1-20.
46. Mutti N S, et al. 2008. A protein from the salivary glands of the pea aphid, Acyrthosiphon pisum, is essential in feeding on a host plant. Proc Natl Acad Sci USA 105:9965-9969.
47. Mutti N S, Park Y, Reeck G R. 2006. RNAi knockdown of a salivary transcript leading to lethality in the pea aphid, Acyrthosiphon pisum. J Insect Sci. 6, 1-7.
48. Nishimura O, Hara Y, Kuraku S. 2017. GVolante for standardizing completeness assessment of genome and transcriptome assemblies. Bioinformatics 33:3635-3637. doi:10.1093/bioinformatics/btx445
49. Niu J, et al. 2019. Topical dsRNA delivery induces gene silencing and mortality in the pea aphid. Pest Manag Sci. 75:2873-2881.
50. Nováková E, Hypša V, Klein J, Foottit R G, von Dohlen C D, Moran N A. 2013. Reconstructing the phylogeny of aphids (Hemiptera: Aphididae) using DNA of the obligate symbiont Buchnera aphidicola. Molecular Phylogenetics and Evolution 68:42-54. doi:10.1016/j.ympev.2013.03.016
51. Parr T. 1940. Asterolecanium variolosum ratzeburg, a gall-forming coccid, and its effect upon the host trees. Yale School of the Environment Bulletin Series, No. 46: 90 pp.
52. Pavlidis P, Zivkovic D, Stamatakis A, Alachiotis N. 2013. SweeD: likelihood-based detection of selective sweeps in thousands of genomes. Mol Biol Evol. 30:2224-2234.
53. Pitino M, Coleman A D, Maffei M E, Ridout C J, Hogenhout S A. 2011. Silencing of aphid genes by dsRNA feeding from plants. PLOS ONE 6:e25709.
54. Paterson S, Vogwill T, Buckling A, Benmayor R, Spiers A J, Thomson N-R, Quail M,
Smith F, Walker D, Libberton B, Fenton A, Hall N, Brockhurst M A. 2010. Antagonistic coevolution accelerates molecular evolution. Nature 464:275-278. doi:10.1038/nature08798
55. Richards S, Gibbs R A, Gerardo N M, Moran N, Nakabachi A, Stem D, Tagu D, Wilson A C C, Muzny D, Kovar C, Cree A, Chacko J, Chandrabose M N, Dao M D, Dinh H H, Gabisi R A, Hines S, Hume J, Jhangian S N, Joshi V, Lewis L R, Liu Y S, Lopez J, Morgan M B, Nguyen N B, Okwuonu G O, Ruiz S J, Santibanez J, Wright R A, Fowler G R, Hitchens M E, Lozado R J, Moen C, Steffen D, Warren J T, Zhang J, Nazareth L V, Chavez D, Davis C, Lee S L, Patel B M, Pu L L, Bell S N, Johnson A J, Vattathil S, Williams R L, Shigenobu S, Dang P M, Morioka M, Fukatsu T, Kudo T, Miyagishima S Y, Jiang H, Worley K C, Legeai F, Gauthier J P, Collin O, Zhang L, Chen H C, Ermolaeva O, Hlavina W, Kapustin Y, Kiryutin B, Kitts P, Maglott D, Murphy T, Pruitt K, Sapojnikov V, Souvorov A, Thibaud-Nissen F, Câmara F, Guigó R, Stanke M, Solovyev V, Kosarev P, Gilbert D, Gabaldón T, Huerta-Cepas J, Marcet-Houben M, Pignatelli M, Moya A, Rispe C, Ollivier M, Quesneville H, Permal E, Llorens C, Futami R, Hedges D, Robertson H M, Alioto T, Mariotti M, Nikoh N, McCutcheon J P, Burke G, Kamins A, Latorre A, Ashton P, Calevro F, Charles H, Colella S, Douglas A E, Jander G, Jones D H, Febvay G, Kamphuis L G, Kushlan P F, Macdonald S, Ramsey J, Schwartz J, Seah S, Thomas G, Vellozo A, Cass B, Degnan P, Hurwitz B, Leonardo T, Koga R, Altincicek B, Anselme C, Atamian H, Barribeau S M, De Vos M, Duncan E J, Evans J, Ghanim M, Heddi A, Kaloshian I, Vincent-Monegat C, Parker B J, Pdrez-Brocal V, Rahbé Y, Spragg C J, Tamames J, Tamarit D, Tamborindeguy C, Vilcinskas A, Bickel R D, Brisson J A, Butts T, Chang C C, Christiaens O, Davis G K, Duncan E J, Ferrier D, Iga M, Janssen R, Lu H L, McGregor A, Miura T, Smagghe G, Smith J, Van Der Zee M, Velarde R, Wilson M, Dearden P, Edwards O R, Gordon K, Hilgarth R S, Rider S D, Srinivasan D, Walsh T K, Ishikawa A, Jaubert-Possamai S, Fenton B, Huang W, Rizk G, Lavenier D, Nicolas J, Smadja C, Zhou J J, Vieira F G, He X L, Liu R, Rozas J, Field L M, Campbell P, Carolan J C, Fitzroy C I J, Reardon K T, Reeck G R, Singh K, Wilkinson T L, Huybrechts J, Abdel-Latief M, Robichon A, Veenstra J A, Hauser F, Cazzamali G, Schneider M, Williamson M M S, Stafflinger E, Hansen K K, Grimmelikhuijzen C J P, Price D R G, Caillaud M, Van Fleet E, Ren Q, Gatehouse J A, Brault V, Monsion B, Diaz J, Hunnicutt L, Ju H J, Pechuan X, Aguilar J, Cortés T, Ortiz-Rivas B, Martinez-Torres D, Dombrovsky A, Dale R P, Davies T G E, Williamson M M S, Jones A, Sattelle D, Williamson S, Wolstenholme A, Cottret L, Sagot M F, Heckel D G, Hunter W, Consortium TIAG. 2010. Genome Sequence of the Pea Aphid Acyrthosiphon pisum. PLoS Biol 8:e1000313-e1000313. doi:10.1371/joumal.pbio.1000313
56. Rodriguez P A, Escudero-Martinez C, Bos J I B. 2017. An aphid effector targets trafficking protein VPS52 in a host-specific manner to promote virulence. Plant Physiol. 173:1892-1903.
57. Rokas A, Holland P W H. 2000. Rare genomic changes as a tool for phylogenetics. Trends Ecol Evol. 15:454-459.
58. Rokas A, Kathirithamby J, Holland P W H. 1999. Intron insertion as a phylogenetic character: the engrailed homeobox of Strepsiptera does not indicate affinity with Diptera. Insect Mol Biol. 8:527-530.
59. Roy S W, Gilbert W. 2005. Rates of intron loss and gain: Implications for early eukaryotic evolution. Proc Natl Acad Sci USA 102:5773-5778. doi:10.1073/pnas.0500383102
60. Ruvinsky A, Watson C. 2007. Intron Phase Patterns in Genes: Preservation and Evolutionary Changes. TOEVOLJ 1:1-14. doi:10.2174/1874404400701010001
61. Schaible G, Lee D J, Kim C S, Schaible G, Garrett L, Lubowski R. 2008. Economic Impacts of the U. S. Soybean Aphid Infestation: A Multi-Regional Competitive Dynamic Analysis. Agricultural and Resource Economics Review 37:227-242. doi:10.1017/S1068280500003026
62. Shakesby A J, et al. 2009. A water-specific aquaporin involved in aphid osmoregulation. Insect Biochem Mol Biol. 39:1-10.
63. Shorthouse J D, Wool D, Raman A. 2005. Gall-inducing insects—Nature's most sophisticated herbivores. Basic and Applied Ecology 6:407-411. doi:10.1016/j.baae.2005.07.001
64. Stem D L, Han, C. 2022. Gene Structure-Based Homology Search Identifies Highly Divergent Putative Effector Gene Family. Genome Biol. Evol.
65. Tekaia F. 2016. Inferring orthologs: open questions and perspectives. Genomics Insights 9:GEI.S37925.
66. Telford M J, Copley R R. 2011. Improving animal phylogenies with genomic data. Trends Genetic. 27:186-195.
67. Thorvaldsdóttir H, Robinson J T, Mesirov J P. 2013. Integrative Genomics Viewer (IGV): High-performance genomics data visualization and exploration. Briefings in Bioinformatics 14:178-192. doi:10.1093/bib/bbs017
68. Vakirlis N, Carvunis A-R, McLysaght A. 2020. Synteny-based analyses indicate that sequence divergence is not the main source of orphan genes. eLife 9:e53500. doi:10.7554/eLife.53500
69. Vellichirammal N N, Gupta P, Hall T A, Brisson J A. 2017. Ecdysone signaling underlies the pea aphid transgenerational wing polyphenism. Proc Natl Acad Sci USA 114:1419-1423.
70. Venkatesh B, Ning Y, Brenner S. 1999. Late changes in spliceosomal introns define clades in vertebrate evolution. Proc Natl Acad Sci USA 96:10267-10271.
71. von Dohlen and N. A. Moran C D. 1995. Molecular phylogeny of the Homoptera a paraphyletic taxon. J Mol Evol 41:211-223.
72. Walczak U, Borowiak-Sobkowiak B, Wilkaniec B. 2019. Tetraneura (Tetraneurella) nigriabdominalis (Hemiptera: Aphidoidea)—a species extending its range in Europe, and morphological comparison with Tetraneura (Tetraneura) ulmi. Entomol Fenn. 28:21-26.
73. Wei H-Y, Ye Y-X, Huang H-J, Chen M-S, Yang Z-X, Chen X-M, Zhang C-X. 2022. Chromosome-level genome assembly for the homed-gall aphid provides insight into interactions between gall-making insect and its host plant. Ecolog and Evlolution. doi.org/10.1002/ece3.8815.
74. Weisenfeld N I, Kumar V, Shah P, Church D M, Jaffe D B. 2017. Direct determination of diploid genome sequences. Genome research 27:757-767. doi:10.1101/gr.235812.118
75. Weisman C M, Murray A W, Eddy S R 2020. Many, but not all, lineage-specific genes can be explained by homology detection failure. PLoS Biol 18:e3000862. doi:10.1371/joumal.pbio.3000862
76. Will T, Tjallingii W F, ThOnnessen A, van Bel A J E. 2007. Molecular sabotage of plant defense by aphid saliva. Proceedings of the National Academy of Sciences of the United States of America 104:10536-10541. doi:10.1073/pnas.0703535104
77. Will T, Vilcinskas A. 2015. The structural sheath protein of aphids is required for phloem feeding. Insect Biochem Mol Biol. 57:34-40.
78. Xie W, He C, Fei Z, Zhang Y. 2020. Chromosome-level genome assembly of the greenhouse whitefly (Trialeurodes vaporariorum Westwood). Molecular Ecology Resources 20:995-1006. doi:10.1111/1755-0998.13159
79. Yang Z. 2007. PAML 4: phylogenetic analysis by maximum likelihood. Mol Biol Evol. 24:1586-1591.

It will be understood that various details of the presently disclosed subject matter can be changed without departing from the scope of the subject matter disclosed herein. Furthermore, the foregoing description is for the purpose of illustration only, and not for the purpose of limitation.

1. A method of identifying a bicycle gene, comprising:
(a) determining for a candidate gene two or more predictor variables selected from the group consisting of:
(i) total gene length (base pair, bp);
(ii) total length (bp) of coding exons;
(iii) first coding exon length (bp);
(iv) last coding exon length (bp);
(v) number of internal exons in phase 0;
(vi) number of internal exons in phase 1;
(vii) number of internal exons in phase 2; and
(viii) mean internal exon length (bp);
(b) applying a bicycle gene classifier including the predictor variables to determine whether the candidate gene is identified as a bicycle gene.

2. The method of claim 1, comprising determining for the candidate gene seven or more of the predictor variables.

3. The method of claim 1, wherein one of the predictor variables is the number of internal exons in phase 2.

4. The method of claim 3, wherein the predictor variables include:
(i) total gene length (base pair, bp);
(ii) total length (bp) of coding exons;
(iii) first coding exon length (bp);
(iv) last coding exon length (bp);
(v) number of internal exons in phase 0;
(vi) number of internal exons in phase 1; and
(vii) number of internal exons in phase 2.

5. The method of claim 1, wherein the bicycle gene classifier is a generalized linear model (GLM).

6. A method of confirming that an identified bicycle gene is a true bicycle gene, comprising:
(a) determined for the identified bicycle gene the presence of one or more conditions, selected from the group consisting of
(i) the gene encodes a protein with an N-terminal secretion signal sequence;
(ii) the gene includes a conserved cysteine-tyrosine-cysteine (CYC) motif when compared to known bicycle genes;
(iii) the gene includes more conserved intron positions than expected by chance, when compared to known bicycle genes; and
(iv) the gene is over-expressed in relevant tissue; and
(b) confirming that the identified bicycle gene is a true bicycle gene when one or more of the conditions is present.

7. A method of identifying a polypeptide encoded by a bicycle gene, comprising:
(a) identifying a bicycle gene according to the method of claim 1, and
(b) determining the amino acid sequence encoded by the nucleotide sequence of the bicycle gene.

8. A polypeptide, comprising the amino acid sequence of the polypeptide identified according to the method of claim 7.

9. A composition, comprising a polypeptide according to claim 8.

10. A polynucleotide, comprising the sequence of a bicycle gene identified according to the method of claim 1.

11. A vector, comprising a polynucleotide according to claim 10.

12. A composition, comprising a vector according to claim 11.

13. A method of modifying a cell, comprising administering to the cell a polynucleotide, polypeptide, composition, or vector according claim 8.

14. The method of claim 13, wherein the cell is a plant cell, an animal cell, a fungal cell, or a bacterial cell.

15. A polypeptide, comprising: one to four cysteine-tyrosine-cysteine (CYC) motifs, attached to one or more flexible or rigid linkers that separate and connect CYC motifs when two or more CYC motifs are present.
